(19) United States
(12) Patent Application Publication
(10) Pub. No.: US 2006/0003950 A1
(43) Pub. Date: Jan. 5, 2006

(54) Method of treating prostatic diseases using a combination of vitamin D analogues and other agents

(75) Inventors: Stephen A. Strugnell, Madison, WI (US); Don Wigington, Middleton, WI (US)

Correspondence Address: MICHAEL BEST & FRIEDRICH, LLP, ONE SOUTH PINCKNEY STREET, P O BOX 1806, MADISON, WI, 53701, US

(73) Assignee: Bone Care International, Inc., Middleton, WI (US)

(21) Appl. No.: 10/881,183

(22) Filed: Jun. 30, 2004

(51) Int. Cl. A61K 31/704; A61K 31/59; A61K 31/28

(52) U.S. Cl. 514/34; 514/167; 514/492

(57) ABSTRACT

The invention provides therapeutic methods for inhibiting, ameliorating or alleviating the hyperproliferative cellular activity of diseases of the prostate, e.g., prostatic cancer and prostatic hyperplasia, which includes administering to a patient in need thereof an active vitamin D analogue and another anticancer agent. Cell differentiation is promoted, induced or enhanced without causing to the patient dose-limiting hypercalcemia and hypercalciuria.

### CROSS-REFERENCE TO RELATED APPLICATIONS

None

### TECHNICAL FIELD

This invention relates generally to a method of treating hyperproliferative prostatic diseases, and in particular, to the use of active compounds of vitamin D in combination with other agents to inhibit the hyperproliferative cellular activity of these diseases and to promote differentiation of the cells.

### BACKGROUND OF THE INVENTION

The prostate gland is found exclusively in male mammals and is subject to certain hyperproliferative diseases. A proliferation of basal and stroma cells of the prostate gland gives rise to benign prostatic hyperplasia which is one common prostate disease. Another common prostate disease is prostate cancer, especially prostatic adenocarcinoma. Adenocarcinoma of the prostate is the most common of the fatal pathophysiological prostate cancers, and typically involves a malignant transformation of epithelial cells in the peripheral region of the prostate gland. Both prostatic hyperplasia and prostate cancer have a high rate of incidence in the aging human male population. Approximately one out of every four males above the age of 55 suffers from a prostate disease of some form or another.

Prostate cancer is currently the second most frequent cause of cancer death after lung cancer among American males. Mortality rates for prostate cancer increase logarithmically with age and are two times higher in U.S. blacks than whites. Internationally, mortality rates are highest in U.S. blacks and in northern Europe and are lowest in Japan. It is projected that by the year 2000, a 90% increase in annual incidence of the disease and a 37% increase in annual mortality rates will be observed. Although prostate cancer may be a relatively indolent neoplasm in the elderly, the overall decrease in life span in patients with this disease is approximately 10 years.

Improvement in the treatment of prostate cancer has centered on early detection. In recent years, screening tests which detect certain proteins or peptides secreted by the prostate gland, i.e., markers, (e.g, prostate-specific antigen (PSA), prostatic acid phosphatase (PAP), prostatic inhibin (PIP)), have increased the power to diagnose this disease in asymptomatic patients.

Treatment of prostate cancer in men under the age of 65 has focused on radical surgery, e.g., prostatectomy, and/or radiotherapy, but the impact of these aggressive approaches on overall survival remains debatable. The approach to treatment of men over the age of 65 historically has been more conservative, and is based on the ablation or control of testosterone production. Such ablation or control is usually achieved by surgical castration, by administration of pituitary gonadotropin inhibitors such as estrogens or luteinizing hormone releasing hormone (LHRH) analogues, or a combination of these treatment methods. Estrogens, such as diethylstilbestrol, are potent inhibitors of the release from the pituitary gland of luteinizing hormone (LH), the gonadotropin that regulates testosterone production, and consequently, estrogen administration can cause a fall in testosterone to castration levels. Maximum suppression of plasma testosterone is typically achieved by a dosage of 3 mg/day of diethylstilbestrol. Other estrogens such as conjugated estrogens are about as equally effective in the lowering of the plasma level as diethylstilbestrol. However, diethylstilbestrol has a poor cardiovascular profile, and death from cardiovascular disease is not uncommon in patients treated with large doses of diethylstilbestrol. Thus, while dosages of up to 3 mg/day of diethylstilbestrol are typically safe, this treatment regime is not indicated for men with preexisting cardiovascular disease.

Prostatic carcinoma often metastasizes to the pelvis and lumbar vertebrae, causing bone loss and associated pain. Hormone manipulation often may result in significant palliation of metastatic prostate cancer, with improvement of bone pain and other disease-associated symptoms. Androgen ablation is, thus, also a major adjunctive therapy in advanced metastatic prostate cancer.

Despite initial improvement on hormonal treatment, a majority of patients with locally unresectable or metastatic disease will eventually fail to respond to further hormonal therapies. A recent study suggests that human prostate cancer cells may cycle between being androgen-independent and androgen-dependent. Such cycling may account for the return of the cancer after initial improvement. In this large group of patients, other forms of treatment, unfortunately, are far less effective. Radiotherapy often may relieve the symptoms of bone pain, but is not curative. Over time, the disease will progress with a fatal outcome.

As noted hereinabove, prostatic hyperplasia is another common hyperproliferative disease of the prostate gland. The disorder affects men over the age of 45 and increases in frequency with age. Prostatic hyperplasia begins in the periurethral region as a localized proliferation and progresses to compress the remaining normal gland. The hyperplasia can compress and obstruct the urethra. Treatment includes surgery, and administration of pituitary gonadotropin inhibitors and/or 5α-reductase enzyme inhibitors.

In another area of physiology and biochemistry, the vitamin D area, extensive research during the past two decades has established important biologic roles for vitamin D apart from its classic role in bone and mineral metabolism. Specific nuclear receptors for 1α,24-dihydroxyvitamin D₃, the hormonally active form of vitamin D, are present in cells from diverse organs not involved in calcium homeostasis.

It has been reported that certain vitamin D compounds and analogues are potent inhibitors of malignant cell proliferation and are inducers/stimulators of cell differentiation. Antiproliferative and differentiating actions of 1α,25-dihydroxyvitamin D₃ and other vitamin D₃ analogues have been reported with respect to prostate cancer cell lines. More recently, an association between vitamin D receptor gene polymorphism and prostate cancer risk has been reported, suggesting that vitamin D receptors may have a role in the development, and possible treatment, of prostate cancer.

### SUMMARY OF THE INVENTION

The present invention provides a method of inhibiting the hyperproliferative activity of human prostatic or neoplastic cells. The method includes use of active vitamin D compounds with other anticancer agents to additively or synergistically inhibit abnormal cell growth and/or promote cell differentiation.

It is anticipated that the vitamin D compounds used in combination with various anticancer drugs can give rise to a significantly enhanced cytotoxic or antineoplastic effect on cancerous cells, thus providing an increased therapeutic effect. Specifically, as a significantly increased growth-inhibitory effect is obtained with the above disclosed combinations utilizing lower concentrations of the anticancer drugs compared to the treatment regimes in which the drugs are used alone, there is the potential to provide therapy wherein adverse side effects associated with the various anticancer drugs are considerably reduced compared to side effects normally observed with the anticancer drugs used alone in larger doses. Alternatively, such combination therapy allows for a greater antineoplastic effect to be derived from a standard dose of anticancer drug, enhancing the effectiveness of the therapy and/or reducing the number of treatments required.

The foregoing is realized in one aspect of the invention utilizing synergistic combinations of 1α,24-dihydroxyvitamin D₂ and various other anticancer agents.

In one aspect the invention provides a method of synergistically inhibiting the growth of human prostatic neoplastic or hyperplastic cells. The method comprises contacting the cells with a first composition which comprises 1α,24-dihydroxyvitamin D₂ and a second composition which comprises carboplatin. Suitably the first and second compositions are provided in therapeutic amounts.

The invention also provides a pharmaceutical combination comprising a first agent which is 1α,24-dihydroxyvitamin D₂ and a second agent which comprises carboplatin. The first and second agents have synergistic properties for inhibiting the hyperproliferative activity of human prostatic neoplastic or hyperplastic cells.

In another aspect, the invention provides the utilization of additive combinations of 1α,24-dihydroxyvitamin D₂ and various other anticancer agents.

In one aspect the invention provides a method of additively inhibiting the growth of human prostatic neoplastic or hyperplastic cells. The method comprises contacting the cells with a first composition which comprises 1α,24-dihydroxyvitamin D₂ and a second composition which comprises an agent selected from the group consisting of carboplatin, doxorubicin, chlorambucil, busulfan, cisplatin, paclitaxel, etoposide, 5-flurouracil, and tamofixen or combinations thereof. Suitably the first and second compositions are provided in therapeutic amounts.

The invention also provides a pharmaceutical combination comprising a first agent which is 1α,24-dihydroxyvitamin D₂ and a second agent selected from the group consisting of carboplatin, doxorubicin, chlorambucil, busulfan, cisplatin, paclitaxel, etoposide, 5-flurouracil, and tamofixen. The first and second agents have additive properties for inhibiting the hyperproliferative activity of human prostatic neoplastic or hyperplastic cells.

Effective amounts of active vitamin D compounds can be administered to patients with cancer or neoplasms. When administered the proliferative activity of the abnormal neoplastic cells is inhibited, reduced, or stabilized, and/or cell differentiation is induced, promoted or enhanced.

The effective amounts of the vitamin D compounds of the invention can be given in an administration protocol in a variety of dose ranges depending on the particular need of the patient. One such suitable dosage range is a range from 0.01 μg to 400 μg. Another suitable dosage range is administered on a daily basis per kilogram of body weight, the dosage ranges being from 0.001 μg/kg/day to 5.0 μg/kg/day. Another dosing regimen calls for a high dosage, generally 10 μg/dose or greater up to 400 μg/dose or greater, given episodically or intermittently. The protocol or dosage regimen provides an improved therapeutic index for active forms of vitamin D analogues compared to administration via conventional regimens. The episodic dosing is also cost effective as less active agent is needed.

Administration of the active vitamin D may be prior to, simultaneous with, or after administration of the other therapeutic agents.

All routes of administration of the active vitamin D or its co-administration with other therapeutic agents are suitable. However, parenteral administration of the active vitamin D compounds, alone or in combination with other agents, provides advantages over other treatment modalities. Parenteral administration bypasses the increased calcemic activity that occurs in the gastrointestinal tract from oral administration and reduces incidence or risk of esophagitis. Parenteral dosing also provides for greater compliance and safety because the drugs are generally administered by a health care professional.

A fuller appreciation of specific adaptations, compositional variations, and physical attributes will be gained upon an examination of the following detailed description of preferred embodiments, taken in conjunction with the appended claims.

### BRIEF DESCRIPTION OF THE DRAWING(S)

FIG. 1 shows the growth inhibition of LNCaP cells by 1α,24-dihydroxyvitamin D₂.

FIG. 2 shows the growth inhibition of LNCaP cells by carboplatin and 1α,24-dihydroxyvitamin D₂.

FIG. 3 shows an isobologram of carboplatin and 1α,24-dihydroxyvitamin D₂ in LNCaP cells.

FIG. 4 shows the growth inhibition of LNCaP cells by caroplatin and 0.01 nM 1α,24-dihydroxyvitamin D₂.

FIG. 5 shows the growth inhibition of LNCaP cells by cisplatin and 1α,24-dihydroxyvitamin D₂.

FIG. 6 shows an isobologram of cisplatin and 1α,24-dihydroxyvitamin D₂ in LNCaP cells.

FIG. 7 shows the growth inhibition of LNCaP cells by cisplatin and 0.01 nM 1α,24-dihydroxyvitamin D₂.

FIG. 8 shows the growth inhibition of LNCaP cells by cisplatin and 0.1 nM 1α,24-dihydroxyvitamin D₂.

FIG. 9 shows the growth inhibition of LNCaP cells by cisplatin and 10 nM 1α,24-dihydroxyvitamin D₂.

FIG. 10 shows the growth inhibition of LNCaP cells by busulfan and 1α,24-dihydroxyvitamin D₂.

FIG. 11 shows an isobologram of busulfan and 1α,24-dihydroxyvitamin D₂ in LNCaP cells.

FIG. 12 shows the growth inhibition of LNCaP cells by busulfan and 0.01 nM 1α,24-dihydroxyvitamin D₂.

FIG. 13 shows the growth inhibition of LNCaP cells by busulfan and 0.1 nM 1α,24-dihydroxyvitamin D₂.

FIG. 14 shows the growth inhibition of LNCaP cells by paclitaxel and 1α,24-dihydroxyvitamin D₂.

FIG. 15 shows an isobologram of paclitaxel and 1α,24-dihydroxyvitamin D₂ in LNCaP cells.

FIG. 16 shows the growth inhibition of LNCaP cells by paclitaxel and 0.3 nM 1α,24-dihydroxyvitamin D₂.

FIG. 17 shows the growth inhibition of LNCaP cells by etoposide and 1α,24-dihydroxyvitamin D₂.

FIG. 18 shows an isobologram of etoposide and 1α,24-dihydroxyvitamin D₂ in LNCaP cells.

FIG. 19 shows the growth inhibition of LNCaP cells by etoposide and 0.01 nM 1α,24-dihydroxyvitamin D₂.

FIG. 20 shows the growth inhibition of LNCaP cells by etoposide and 0.1 nM 1α,24-dihydroxyvitamin D₂.

FIG. 21 shows the growth inhibition of LNCaP cells by etoposide and 1 nM 1α,24-dihydroxyvitamin D₂.

FIG. 22 shows the growth inhibition of LNCaP cells by 5-fluorouracil and 1α,24-dihydroxyvitamin D₂.

FIG. 23 shows an isobologram of 5-fluorouracil and 1α,24-dihydroxyvitamin D₂ in LNCaP cells.

FIG. 24 shows the growth inhibition of LNCaP cells by 5-fluorouracil and 0.01 nM 1α,24-dihydroxyvitamin D₂.

FIG. 25 shows the growth inhibition of LNCaP cells by tamoxifen and 1α,24-dihydroxyvitamin D₂.

FIG. 26 shows an isobologram of tamoxifen and 1α,24-dihydroxyvitamin D₂ in LNCaP cells.

FIG. 27 shows the growth inhibition of LNCaP cells by tamoxifen and 0.01 nM 1α,24-dihydroxyvitamin D₂.

FIG. 28 shows the growth inhibition of LNCaP cells by tamoxifen and 0.1 nM 1α,24-dihydroxyvitamin D₂.

FIG. 29 shows the growth inhibition of LNCaP cells by doxorubicin and 1α,24-dihydroxyvitamin D₂.

FIG. 30 shows an isobologram of doxorubicin and 1α,24-dihydroxyvitamin D₂ in LNCaP cells.

FIG. 31 shows the growth inhibition of LNCaP cells by doxorubicin and 0.01 nM 1α,24-dihydroxyvitamin D₂.

FIG. 32 shows the growth inhibition of LNCaP cells by doxorubicin and 0.1 nM 1α,24-dihydroxyvitamin D₂.

FIG. 33 shows the growth inhibition of LNCaP cells by doxorubicin and 1 nM 1α,24-dihydroxyvitamin D₂.

FIG. 34 shows combination index values for chemotherapeutic agents and 1α,24-dihydroxyvitamin D₂ combinations in LNCaP cells.

Before the embodiments of the invention are explained in detail, it is to be understood that the phraseology and terminology used herein are for the purpose of description and should not be regarded as limiting. The use of “including”, “having” and “comprising” and variations thereof herein is meant to encompass the items listed thereafter and equivalents thereof as well as additional items and equivalents thereof.

### DETAILED DESCRIPTION OF THE INVENTION

The present invention includes an effective method for the treatment of neoplastic and hyperplastic diseases. Particularly, the present invention relates to therapeutic methods for additively or synergistically inhibiting the growth of human prostatic neoplastic or hyperplastic cells by the use of combinations of vitamin D analogs and other therapeutic agents.

The methods of the present invention include administering to cells, a patient, or a subject, a first composition which comprises a vitamin D analog, and a second composition which comprises a therapeutic agent. The first and second compositions additively or synergistically inhibit the growth of human prostatic neoplastic or hyperplastic cells. Suitably, the active vitamin D analogs include 1α,24-dihydroxyvitmin D₂.

As used herein the term “additively inhibits” means that the total inhibitory effect of the agents administered is approximately the sum of their individual inhibitory effects.

As used herein the term “synergistically inhibits” means that the total inhibitory effect of the agents administered is greater than the sum of the individual inhibitory effects of the agents.

It is known that vitamin D₃ must be hydroxylated in the C-1 and C-25 positions before it is activated, i.e., before it will produce a biological response. A similar metabolism appears to be required to activate other forms of vitamin D, e.g., vitamin D₂ and vitamin D₄. Therefore, as used herein, the term “activated vitamin D” or “active vitamin D” is intended to refer to a vitamin D compound or analogue that has been hydroxylated in at least the C-1 position of the A ring of the molecule and either the compound itself or its metabolites in the case of a prodrug, such as 1α-hydroxyvitamin D₂, binds the vitamin D receptor (VDR). Vitamin D compounds which are hydroxylated only in the C-1 position are referred to herein as “prodrugs.” Such compounds undergo further hydroxylation in vivo and their metabolites bind the VDR.

Also, as used herein, the term “lower” as a modifier for alkyl, alkenyl acyl, or cycloalkyl is meant to refer to a straight or branched, saturated or unsaturated hydrocarbon radical having 1 to 4 carbon atoms. Specific examples of such hydrocarbon radicals are methyl, ethyl, propyl, isopropyl, butyl, isobutyl, t-butyl, ethenyl, propenyl, butenyl, isobutenyl, isopropenyl, formyl, acetyl, propionyl, butyryl or cyclopropyl. The term “aromatic acyl” is meant to refer to a unsubstituted or substituted benzoyl group.

As used herein, the term “hydrocarbon moiety” refers to a lower alkyl, a lower alkenyl, a lower acyl group or a lower cycloalkyl, i.e., a straight or branched, saturated or unsaturated C₁-C₄ hydrocarbon radial.

The term “contacting” is used herein interchangeably with the following: combined with, added to, mixed with, passed over, incubated with etc. Moreover, the compounds of present invention can be “administered” by any conventional method such as, for example, parenteral, oral, topical and inhalation routes as described herein.

Thus, the present invention includes a method of treating malignant prostatic cells as well as other hyperproliferative prostatic cells, (i.e., inhibiting or reducing their hyperproliferative activity and/or inducing and enhancing their differentiation) with an effective amount of a vitamin D analogs, co-administered with various cytotoxic agents such that the combination of the vitamin D analog and cytotoxic agent provides additive or synergistic effects in the inhibition of hyperproliferative activity of the prostatic cells, i.e., the cells are treated and contacted with both agents.

The term “co-administration” is meant to refer to a combination therapy by any administration route in which two or more agents are administered to cells, to a patient or to a subject. Co-administration of agents may be referred to as combination therapy or combination treatment. In respect to treatment of patients, the agents may be the same dosage formulations or separate formulations. For combination treatment with more than one active agent, where the active agents are in separate dosage formulations, the active agents can be administered concurrently, or they each can be administered at separately staggered times. The agents may be administered simultaneously or sequentially, as along as they are given in a manner sufficient to allow both agents to achieve effective concentrations in the body. The agents may be administered by different routes, e.g., one agent may be administered intravenously while a second agent is administered intramuscularly, intravenously or orally. The agents also may be in an admixture, as, for example, in a single tablet.

In time-sequential co-administration, one agent may directly follow administration of the other or the agents may be give episodically, i.e., one can be given at one time followed by the other at a later time, e.g., within a week. An example of a suitable co-administration regimen is where an active vitamin D compound is administered from 0.5 to 7 days prior to administration of a cytotoxic or other therapeutic agent.

Suitable cytotoxic agents include busulfan, 5-fluorouracil, paclitaxel, tamoxifen, cisplatin, carboplatin, doxorubicin (adriamycin), chlorambucil, etoposide, melphalan (Alkeran™), estramustine (Emcyt™), hydroxyurea, hydroxycarbamide (Hydrea™), mitomycin, idarubicin, methotrexate, daunomycin and prednimustine. Use of an active vitamin D analog in combination with various anticancer drugs can give rise to a significantly enhanced cytotoxic effect on cancerous cells, thus providing an increased therapeutic effect. Specifically, as a significantly increased growth-inhibitory effect is obtained with the above disclosed combinations utilizing lower concentrations of the anticancer drugs compared to the treatment regimes in which the drugs are used alone, there is the potential to provide therapy wherein adverse side effects associated with the anticancer drugs are considerably reduced than normally observed with the anticancer drugs used alone in larger doses. Possible dose ranges of these co-administered second anticancer agents are listed below in Table 1

TABLE 1

| Agent | Dose Ranges per Day | |
|---|---|---|
| Busulfan | 0.01-0.1 | mg/kg |
| Carboplatin | 7.8 | mg/kg |
| Cisplatin | 0.4-2.6 | mg/kg |
| Chlorambucil | 0.1-0.4 | mg/kg |
| Daunomycin | 0.65-1.0 | mg/kg |
| Doxorubicin (Adriamycin) | 1.3-1.6 | mg/kg |
| Estramustine (Emcyt) | 14 | mg/kg |
| Etoposide | 0.75-2.2 | mg/kg |
| 5-Fluorouracil | 10-25 | mg/kg |
| Hydroxyurea | 20-80 | mg/kg |
| Hydroxycarbamide (Hydrea) | 7 | mg/kg |
| Idarubicin | 0.26 | mg/kg |
| Melphalan (Alkeran) | 0.08-0.2 | mg/kg |
| Methotrexate | 0.03-260 | mg/kg |
| Mitomycin | 0.1-0.5 | mg/kg |
| Paclitaxel | 2.9-3.8 | mg/kg |
| Prednimustine | 2.15 | mg/kg |
| Tamoxifen | 0.14 | mg/kg |

Depending on the combination of the particular vitamin D analog and second anticancer agent, and other factors such as concentration and amount of the agents, additive, synergistic or antagonistic inhibiting growth effects on human prostatic or hyperplastic cells can be found.

1α,24-dihydroxyvitamin D₂ when utilized in combination with the agent carboplatin can synergistically inhibits the growth of human prostatic, neoplastic or hyperplastic cells. 1α,24-dihydroxyvitamin D₂ can also be utilized with a second composition to additively inhibit the growth of human prostatic, neoplastic or hyperplastic cells. Such second compositions include carboplatin, doxorubicin, chlorambucil, busulfan, cisplatin, paclitaxel, etoposide, 5-flurouracil, and tamofixen or combinations thereof.

The effective amounts of vitamin D compound can be given in an administration protocol in a variety of dose ranges depending on the particular need of the patient. One such suitable dose range is administered on a daily basis per kilogram of body weight, the dose ranges being from 0.001 μg/kg/day to 5.0 μg/kg/day. Another dosing regimen calls for a high dosage, generally 10 μg/dose or greater up to 400 μg/dose or greater, given episodically or intermittently. Such protocol, or dosage regimens provide an improved therapeutic index for active forms of vitamin D analogues compared to administration via conventional regimens. The episodic dosing is also cost effective as less active agent is needed.

In an episodic dosing regimen, each single dose is sufficient to upregulate vitamin D hormone receptors in target cells. It is believed that continuous dosing is not required because the binding and upregulation by vitamin D compounds is sufficient to initiate the cascade of intracellular metabolic processes occurring with receptor binding. Intermittent dosing reduces the risk of hypercalcemia, and thus, the method in accordance with the present invention can be used to treat hyperproliferative diseases by administering any active vitamin D compound. At the same time, it is contemplated that the risk of hypercalcemia can be further mitigated if the active vitamin D compound is a hypocalcemic active vitamin D compound.

It is further believed that the intermittent dose regimen can be used to effect any therapeutic effect that is attributable to active vitamin D., e.g., antiproliferative activity, reduction of loss of bone mass, etc. In regard to antiproliferative activity, the value of the intermittent dosing is that antihyperproliferative activity and upregulation of vitamin D receptors occurs with a single dose without the side effects of hypercalcemia and hypercalciuria that occur with recurrent daily dosing.

The episodic dose can be a single dose or, optionally, divided into 2-4 subdoses which, if desired, can be given, e.g., twenty minutes to an hour apart until the total dose is given. The compounds in accordance with the present invention are administered in an amount that raises serum vitamin D levels to a supraphysiological level for a sufficient period of time to induce differentiation or regression of a tumor or neoplasm without causing hypercalcemia or with substantially reduced the risk of hypercalcemia. The properties of the hypocalcemic vitamin D compounds are particularly beneficial in permitting such supraphysiologic levels.

As described above, the present invention relates to a method of co-administration of active vitamin D compounds with an anticancer or antineoplastic or cytotoxic agent. Therapeutic antihyperproliferative benefits are achieved with intermittent dosing of active vitamin D with cytotoxic, i.e., other chemotherapeutic or antineoplastic, agents. Many antineoplastic or cytotoxic agents must be delivered through a parenteral route of administration, and thus, a protocol of injectable active vitamin D and antineoplastic agent can be set up on a routine basis. The co-administration of active vitamin D and antineoplastic agents can be prior to, after, or simultaneous with each other. However, it is believed that the prior administration of active vitamin D with the later episodic administration of a cytotoxic or antineoplastic agent is of benefit. For example, a high dose active vitamin D upregulates the receptors, and primes and promotes cell differentiation. Such upregulation and priming, potentially permits less cytotoxic or antineoplastic agent than would typically be required if the cytotoxic agent were administered alone.

Those of ordinary skill in the art will readily optimize effective doses and co-administration regimens (as described hereinbelow) as determined by good medical practice and the clinical condition of the individual patient. Regardless of the manner of administration, it will be appreciated that the actual preferred amounts of active compound in a specific case will vary according to the efficacy of the specific compound employed, the particular compositions formulated, the mode of application, and the particular situs and organism being treated. For example, the specific dose for a particular patient depends on age, body weight, general state of health, on diet, on the timing and mode of administration, on the rate of excretion, and on medicaments used in combination and the severity of the particular disorder to which the therapy is applied. Dosages for a given patient can be determined using conventional considerations, e.g., by customary comparison of the differential activities of the subject compounds and of a known agent, such as by means of an appropriate conventional pharmacological protocol. A physician of ordinary skill can readily determine and prescribe the effective amount of the drug required to counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug. The dosage of active ingredient in the compositions of this invention may be varied; however, it is necessary that the amount of the active ingredient be such that an efficacious dosage is obtained. The active ingredient is administered to patients (animal and human) in need of treatment in dosages that will provide optimal pharmaceutical efficacy.

The pharmacologically active compounds of this invention can be processed in accordance with conventional methods of pharmacy to produce medicinal agents for administration to patients, e.g., mammals including humans. The vitamin D analogs and cytotoxic agents can be co-administered separately at the same time, at proximate times, or can be delivered simultaneously in an admixture. Both the vitamin D analog, the cytotoxic agent, or the admixed combination of the two can be employed in admixtures with conventional excipients, e.g., pharmaceutically acceptable carrier substances suitable for enteral (e.g., oral) or parenteral application which do not deleteriously react with the active compounds.

Active vitamin D compounds can be formulated in pharmaceutical compositions in a conventional manner using one or more conventional excipients, which do not deleteriously react with the active compounds, e.g., pharmaceutically acceptable carrier substances suitable for enteral administration (e.g., oral), parenteral, topical, buccal or rectal application, or by administration by inhalation or insufflation (e.g., either through the mouth or the nose)

Generally, acceptable carriers for pharmaceutical formulation include, but are not limited to, water, salt solutions, alcohols, gum arabic, vegetable oils (e.g., almond oil, corn oil, cottonseed oil, peanut oil, olive oil, coconut oil), mineral oil, fish liver oils, oily esters such as Polysorbate 80, polyethylene glycols, gelatine, carbohydrates (e.g., lactose, amylose or starch), magnesium stearate, talc, silicic acid, viscous paraffin, fatty acid monoglycerides and diglycerides, pentaerythritol fatty acid esters, hydroxy methylcellulose, polyvinyl pyrrolidone, etc.

Of particular interest is the parenteral, e.g., injectable, dosage form. Using the parenteral route of administration allows for bypass of the first pass of active vitamin D compound through the intestine, thus avoiding stimulation of intestinal calcium absorption, and further reduces the risk of esophageal irritation which is often associated with high dose oral administration. Because an injectable route of administration is typically done by a health care professional, the dosing can be more effectively controlled as to precise amount and timing. Parenteral administration suitably includes subcutaneous, intramuscular, or intravenous injection, nasopharyngeal or mucosal absorption, or transdermal absorption. Where indicated, the vitamin D compositions may also be given by direct injection into the tumor by intraarterial delivery or delivery via the portal vein.

The injectable compositions may take such forms as sterile suspensions, solutions, or emulsions in oily vehicles (such as coconut oil, cottonseed oil, sesame oil, peanut oil or soybean oil) or aqueous vehicles, and may contain various formulating agents. Alternatively, the active ingredient may be in powder (lyophilized or non-lyophilized) form for reconstitution at the time of delivery with a suitable vehicle, such as sterile water. In injectable compositions, the carrier is typically sterile, pyrogen-free water, saline, aqueous propylene glycol, or another injectable liquid, e.g., peanut oil for intramuscular injections. Also, various buffering agents, preservatives, suspending, stabilizing or dispensing agents, surface-active agents and the like can be included. Aqueous solutions may be suitably buffered, if necessary, and the liquid diluent first rendered isotonic with sufficient saline or glucose. Aqueous solutions are especially suitable for intravenous, intramuscular, subcutaneous and intraperitoneal injection purposes. In this connection, the sterile aqueous media employed are all readily obtainable by standard techniques well-known to those skilled in the art. The oily solutions are suitable for intra-articular, intramuscular and subcutaneous injection purposes. The preparation of all these solutions under sterile conditions is readily accomplished by standard pharmaceutical techniques well-known to those skilled in the art. Additionally, it is also possible to administer the compounds of the present invention topically when treating pathological conditions of the skin, and this may suitably be done by way of creams, jellies, gels, pastes, ointments and the like, in accordance with standard pharmaceutical practice.

The compounds formulated for parenteral administration by injection may be administered, by bolus injection or continuous infusion. Formulations for injection may be conveniently presented in unit dosage form, e.g., in ampoules or in multi-dose, multi-use containers, with an added preservative.

In addition to the formulations described previously, the compounds may also be formulated as a depot preparation. Such long acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion exchange resins, or as sparingly soluble derivatives, e.g., a sparingly soluble salt.

Although it is considered that episodic parenteral administration of active vitamin D is highly beneficial, it is also contemplated within the scope of the present invention that enteral dosing, e.g., oral administration, can also be of benefit. Thus, episodic enteral dosing of high dose active vitamin D is also considered of benefit in achieving the upregulation of cell receptors.

For enteral application, particularly suitable are tablets, dragees, liquids, drops, suppositories, lozenges, powders, or capsules. A syrup, elixir, or the like can be used if a sweetened vehicle is desired. For oral administration, the pharmaceutical compositions may take the form of, for example, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art.

Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may also be suitably formulated to give controlled release of the active compound. Many controlled release systems are known in the art.

For buccal administration, the compositions may take the form of tablets, lozenges or absorption wafers formulated in conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of e.g. gelatin, for use in an inhaler or insufflator may be formulated containing a powder mix of the active compound and a suitable powder base such as lactose or starch.

The compounds may also be formulated in rectal or vaginal compositions such as suppositories containing conventional suppository bases or retention enemas. These compositions can be prepared by mixing the active ingredient with a suitable non-irritating excipient which is solid at room temperature (for example, 10° C. to 32° C.) but liquid at the rectal temperature, and will melt in the rectum or vagina to release the active ingredient. Such materials are polyethylene glycols, cocoa butter, other glycerides and wax. To prolong storage life, the composition advantageously may include an antioxidant such as ascorbic acid, butylated hydroxyanisole or hydroquinone.

The compositions may, if desired, be presented in a pack or dispenser device which may contain one or more unit dosage forms containing the active ingredient. The pack may, for example, comprise metal or plastic foil, such as a blister pack. The pack or dispenser device may be accompanied by instructions for administration.

The pharmaceutical preparations can be sterilized and, if desired, be mixed with auxiliary agents, e.g., lubricants, preservatives, stabilizers, wetting agents, emulsifiers, salts for influencing osmotic pressure, buffers, coloring, flavoring and/or one or more other active compounds, for example, conjugated estrogens or their equivalents, anti-estrogens, calcitonin, bisphosphonates, calcium supplements, cobalamin, pertussis toxin, boron, antineoplastic agents and antihypercalcemic agents.

The present invention is further explained by the following examples which should not be construed by way of limiting the scope of the present invention.

### VDR Binding Analyses

### EXAMPLE 1

### 1α,24-dihydroxyvitamin D₂ [1α,24-(OH)₂D₂]

The affinity of 1α,24-(OH)₂D₂ for the mammalian vitamin D receptor (VDR) was assessed using a commercially available kit of bovine thymus VDR and standard 1,25-(OH)₂D₃ solutions from Incstar (Stillwater, Minn.). The half-maximal binding of chemically synthesized 1α,24-(OH)₂D₂ was approximately 150 pg/ml whereas that of 1α,25-(OH)₂D₃ was 80 pg/ml. Thus, the 1α,24-(OH)₂D₂ had a very similar affinity for bovine thymus VDR as did 1α,25-(OH)₂D₃, indicating that 1α,24-(OH)₂D₂ has potent biological activity.

### EXAMPLE 2

### 1α,24-dihydroxyvitamin D₂ [1α,24-(OH)₂D₂]

VDR binding of vitamin D compounds by prostate cells is demonstrated using the techniques of Skowronski et al., 136 Endocrinology (1995) 20-26, which is incorporated herein by reference. Prostate-derived cell lines are cultured to near confluence, washed and harvested by scraping. Cells are washed by centrifugation, and the cell pellet resuspended in a buffered salt solution containing protease inhibitors. The cells are disrupted by sonication while cooling on ice. The supernatant obtained from centrifuging the disrupted cells at 207,000×g for 35 min at 4° C. is assayed for binding. 200 μL of soluble extract, (1-2 mg protein/ml supernatant) is incubated with a 1 nM 3H-1α,25-(OH)₂D₃ and increasing concentrations of 1α,24-(OH)₂-D₂ (0.01-100 nM) for 16-20 hr at 4° C. Bound and free hormones are separated with hydroxylapatite using standard procedures. Specific binding is calculated by subtracting nonspecific binding obtained in the presence of a 250-fold excess of nonradioactive 1α,25-(OH)₂D₃ from the total binding measured. The results demonstrate that 1α,24-(OH)₂D₂ has strong affinity for prostate VDR, indicating that 1α,24-(OH)₂D₂ has potent biological activity in respect of prostate cells.

### Gene Expression

### EXAMPLE 3

### 1α,24(S)-dihydroxyvitamin D₂ and 1α,24(R)-dihydroxy-vitamin D₂ [1α,24(S)—(OH)₂D₂ and 1α,24(R)—(OH)₂D₂]

Using the plasmids pSG5-hVDR1/3, a vitamin D receptor (VDR)-expressing plasmid, and p(CT4)4TKGH, a plasmid containing a Growth Hormone (GH) gene, under the control of a vitamin D-responsive element (VDRE), experiments were conducted to explore the ability of 1α,24-(OH)₂D₂ to induce vitamin D-dependent growth hormone acting as a reporter gene compared to that of 1α,25-(OH)₂D₃. Cells in culture were co-transfected into Green monkey kidney, COS-1 cells with these two plasmids. One plasmid contained the gene for Growth Hormone (GH) under the control of the vitamin D responsive element (VDRE) and the other plasmid contained the structural gene for the vitamin D receptor (VDR). These transfected cultures were incubated with 1α,24-(OH)₂D₂ or 1α,25-(OH)₂D₃, and the production of growth hormone was measured.

As shown in Table 2, both 1α,24(S)—(OH)₂D₂ and its epimer, 1α,24(R)—(OH)₂D₂, had significantly more activity in this system than 25-OH-D₃, with 1α,24(S)—(OH)₂D₂ having nearly the same activity as 1α,25-(OH)₂D₃.

TABLE 2
Vitamin D-Inducible Growth Hormone Production
In Transfected COS-1 Cells
Vitamin D Inducible Growth Hormone Production

| | | | Net vitamin D |
|---|---|---|---|
| | | Total GH | inducible |
| | Molar | Production* | GH-production |
| Inducer | Concentration | (ng/ml) | (ng/ml) |
| Ethanol | | 44 | 0 |
| 25-OH-D₃ | 1 × 10⁻⁷ | 245 | 201 |
| | 1 × 10⁻⁶ | 1100 | 1056 |
| | 1 × 10⁻⁵ | 775 | 731 |
| 1α,25-(OH)₂D₃ | 1 × 10⁻¹⁰ | 74 | 30 |
| | 1 × 10⁻⁹ | 925 | 881 |
| | 1 × 10⁻⁸ | 1475 | 1441 |
| 1α,24(S)—(OH)₂D₂ | 5 × 10⁻¹⁰ | 425 | 381 |
| | 5 × 10⁻⁹ | 1350 | 1306 |
| | 5 × 10⁻⁸ | 1182 | 1138 |
| 1α,24(R)—(OH)₂D₂ | 1 × 10⁻⁹ | 80 | 36 |
| | 1 × 10⁻⁸ | 1100 | 1056 |
| | 1 × 10⁻⁷ | 1300 | 1256 |

*Averages of duplicate determinations

### Inhibition of Prostate Cell Proliferation

### EXAMPLE 4

### 1α,24-dihydroxyvitamin D₂ [1α,24-(OH)₂D₂]

Inhibition of cell proliferation is demonstrated using the techniques of Skowronski et al., 132 Endocrinology (1993) 1952-1960 and 136 Endocrinology (1995) 20-26, both of which are incorporated herein by reference. The cell lines, LNCaP and PC-3, which are derived from human prostate adenocarcinoma, are seeded in six-well tissue culture plates at a density of about 50,000 cells/plate. After the cells have attached and stabilized, about 2-3 days, the medium is replenished with medium containing vehicle or the active vitamin D analogue 1α,24-(OH)₂D₂, at concentrations from 10⁻¹¹ M to 10⁻⁷ M. Medium containing test analogue or vehicle is replaced every three days. After 6-7 days, the medium is removed, the cells are rinsed, precipitated with cold 5% trichloroacetic acid, and washed with cold ethanol. The cells are solubilized with 0.2 N sodium hydroxide, and the amount of DNA determined by standard procedures. The results show that cultures incubated with 1α,24-(OH)₂D₂ in accordance with the present invention have significantly fewer cells than the control cultures.

### Stimulation of Prostate Cell Differentiation

### EXAMPLE 5

### 1α,24-dihydroxyvitamin D₂ [1α,24-(OH)₂D₂]

Using the techniques of Skowronski et al., 132 Endocrinology (1993) 1952-1960 and 136 Endocrinology (1995) 20-26, both of which are incorporated herein by reference, cells of the cell line, LNCaP, which is derived from a human metastatic prostate adenocarcinoma and known to express PSA, are seeded in six-well tissue culture plates at a density of about 50,000 cells/plate. After the cells have attached and stabilized, about 2-3 days, the medium is replenished with medium containing vehicle or the active vitamin D analogue, 1α,24-(OH)₂D₂, at concentrations from 10⁻¹¹ M to 10⁻⁷ M. After 6-7 days, the medium is removed and stored at −20° C. for prostate specific antigen (PSA) analysis.

The cells from parallel cultures are rinsed, precipitated, and the amount of DNA determined by standard procedures. PSA is measured by standard known methods. Cultures incubated with 1α,24-(OH)₂D₂ have significantly more PSA than control cultures when expressed as mass of PSA/cell.

### Clinical Studies

### EXAMPLE 6

### 1α,24-dihydroxy vitamin D₂ [1α,24-(OH)₂D₂]

Patients with advanced androgen-independent prostate cancer participate in an open-labeled study of 1α,24-(OH)₂D₂. Qualified patients are at least 40 years old, exhibit histologic evidence of adenocarcinoma of the prostate, and present with progressive disease which had previously responded to hormonal intervention(s). On admission to the study, patients begin a course of therapy with oral 1α,24-(OH)₂D₂ lasting 26 weeks, while discontinuing any previous use of calcium supplements, vitamin D supplements, and vitamin D hormone replacement therapies. During treatment, the patients are monitored at regular intervals for: (1) hypercalcemia, hyperphosphatemia, hypercalciuria, hyperphosphaturia and other toxicity; (2) evidence of changes in the progression of metastatic disease; and (3) compliance with the prescribed test drug dosage.

The study is conducted in two phases. During the first phase, the maximal tolerated dosage (MTD) of daily oral 1α,24-(OH)₂D₂ is determined by administering progressively higher dosages to successive groups of patients. All doses are administered in the morning before breakfast. The first group of patients is treated with 25.0 μg of 1α,24-(OH)₂D₂. Subsequent groups of patients are treated with 50.0, 75.0 and 100.0 μg/day. Dosing is continued uninterrupted for the duration of the study unless serum calcium exceeds 11.6 mg/dL, or other toxicity of grade 3 or 4 is observed, in which case dosing is held in abeyance until resolution of the observed toxic effect(s) and then resumed at a level which has been decreased by 10.0 μg.

Results from the first phase of the study show that the MTD for 1α,24-(OH)₂D₂ is above 20.0 μg/day, a level which is 10- to 40-fold higher than can be achieved with 1α,25-(OH)₂D₃. Analysis of blood samples collected at regular intervals from the participating patients reveal that the levels of circulating 1α,24-(OH)₂D₂ increase proportionately with the dosage administered, rising to maximum levels well above 100 pg/mL at the highest dosages, and that circulating levels of 1α,25-(OH)₂D₃ are suppressed, often to undetectable levels. Serum and urine calcium are elevated in a dose responsive manner. Patients treated with the MTD of 1α,24-(OH)₂D₂ for at least six months report that bone pain associated with metastatic disease is significantly diminished.

During the second phase, patients are treated with 1α,24-(OH)₂D₂ for 24 months at 0.5 and 1.0 times the MTD. After one and two years of treatment, CAT scans, X-rays and bone scans used for evaluating the progression of metastatic disease show stable disease or partial remission in many patients treated at the lower dosage, and stable disease and partial or complete remission in many patients treated at the higher dosage.

### CO-ADMINISTRATION OF VITAMIN D ANALOGS AND CYTOTOXIC AGENTS

### EXAMPLE 7

### Co-Administration of Vitamin D Analogs and Cytotoxic Agents Protocol

Vitamin D agents are tested for synergistic and additive interactions with anticancer drugs on human LNCaP prostate cancer cell lines. LNCap cells were plated in 96-well plates in triplicate and allowed to grow 48 hours. The medium was removed and replaced with medium containing vehicle (Ethanol), vitamin D compounds (1,24(OH)₂D_{2,} 1,25(OH)₂D₃ or 1,25(OH)₂D₂), and/or chemotherapeutic agents (busulfan, 5-fluorouracil, paclitaxel, tamoxifen, cisplatin, carboplatin, doxorubicin, chlorambucil, or etoposide). Cells were allowed to grow for an additional 6 days with media changed on day 3. Cell number was then determined by a colorimetric MTS assay and expressed as a % of change from control cells grown in vehicle only. ID₃₀ values (dose required to inhibit proliferation by 30%) were calculated to compare growth inhibitory effects of the compounds alone and in combination. Isobologram analysis was used to characterize the interaction between vitamin D compounds and anti-cancer drugs as synergistic, additive, or antagonistic.

### EXAMPLE 8

### Growth Inhibition of LNCaP Cells by 1,24(OH)₂D₂ Alone

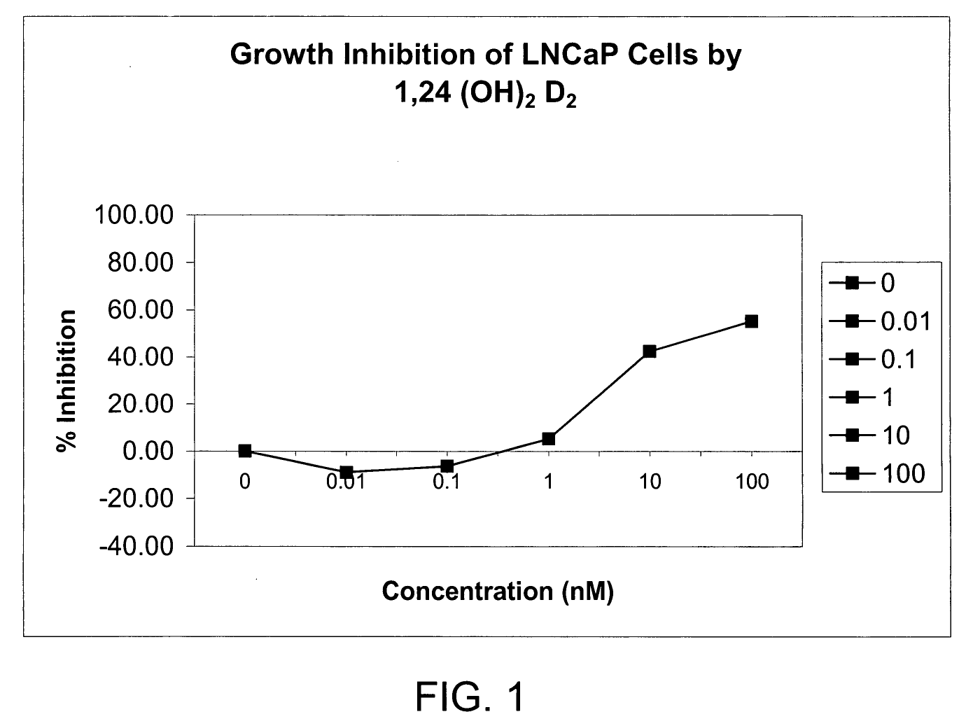

LNCap cells were plated in 96-well plates in triplicate and allowed to grow 48 hours. The medium was removed and replaced with medium containing vehicle (Ethanol) and 1,24(OH)₂D₂ in various concentrations. Cells were allowed to grow for an additional 6 days with media changed on day 3. Cell number was then determined by a colorimetric MTS assay and expressed as a % of change from control cells grown in vehicle only. The growth inhibition of the cells by 1,24(OH)₂D₂ are shown in FIG. 1.

### EXAMPLE 9

### Growth Inhibition of LNCaP Cells by Carboplatin and 1,24(OH)₂D₂

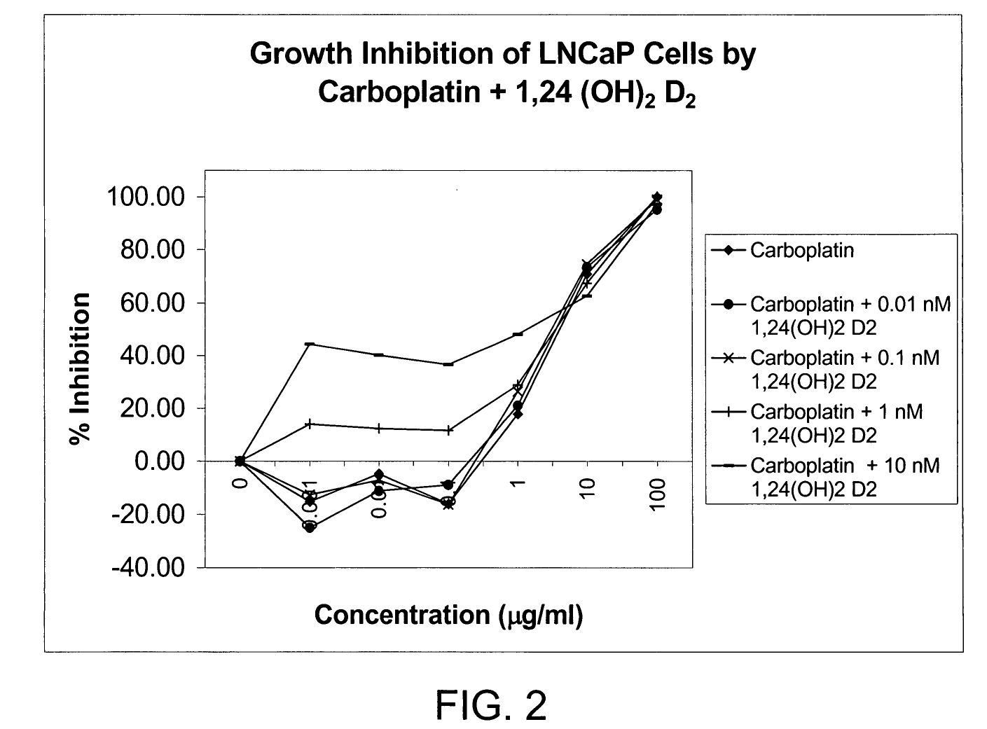

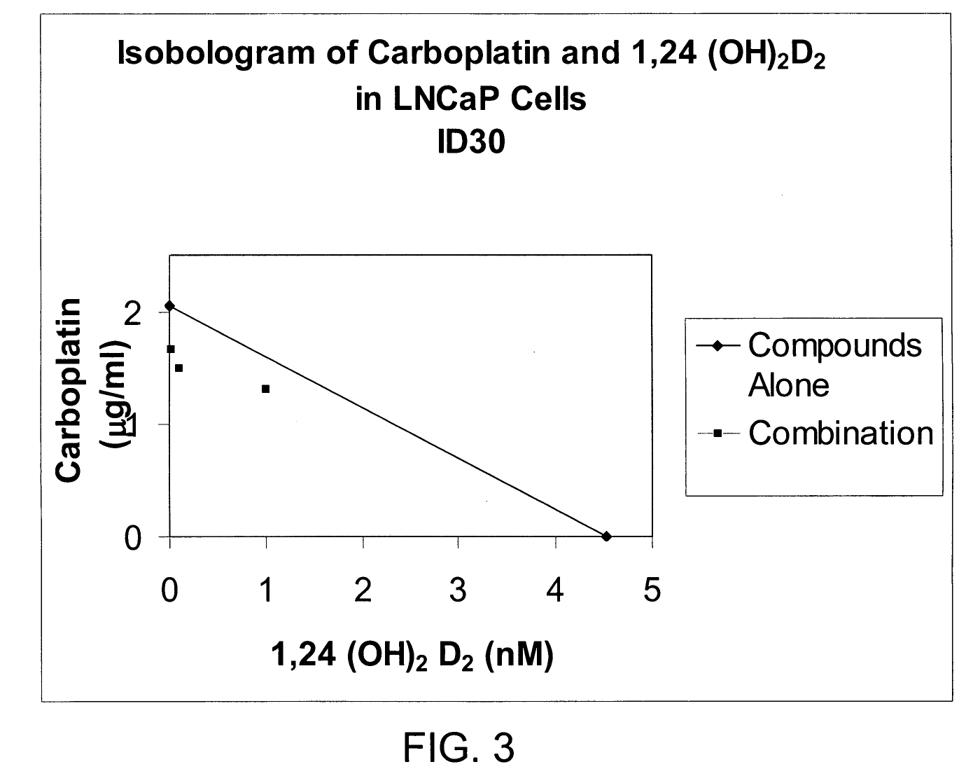

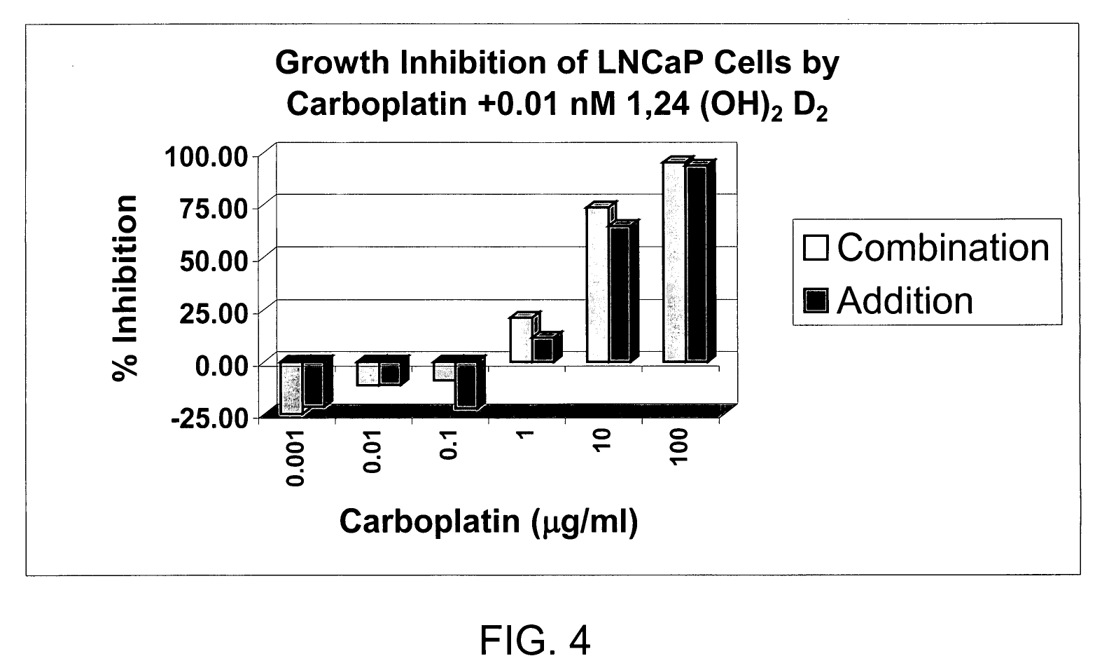

LNCap cells were plated in 96-well plates in triplicate and allowed to grow 48 hours. The medium was removed and replaced with medium containing vehicle (Ethanol), 1,24(OH)₂D₂ in various concentrations, and carboplatin in various concentrations. Cells were allowed to grow for an additional 6 days with media changed on day 3. Cell number was then determined by a colorimetric MTS assay and expressed as a % of change from control cells grown in vehicle only. FIG. 2 shows the percent inhibition of LNCaP cells of carboplatin alone or in combination with various concentrations of 1,24(OH)₂D₂. ID30 values (dose required to inhibit proliferation by 30%) were calculated to compare growth inhibitory effects of the compounds alone and in combination. Isobologram analysis was used to characterize the interaction between 1,24(OH)₂D₂ and carboplatin as synergistic, additive, or antagonistic. The isobologram is shown in FIG. 3, and shows that carboplatin in the concentration range of about 0 to 2 μg/mL when combined with 1,24(OH)₂D₂ has a synergistic effect. This effect can also be seen in FIG. 4. The addition columns show the amount of inhibition predicted if the combination of carboplatin and 1,24(OH)₂D₂ simply had an additive effect on each other. The growth inhibition chart shows that the combination of carboplatin in concentrations of 1 μg/mL, 10 μg/mL, and 100 μg/mL with 0.01 nM of 1,24(OH)₂D₂ produces synergistic growth inhibition.

### EXAMPLE 10

### Growth Inhibition of LNCaP Cells by Cisplatin and 1,24(OH)₂D₂

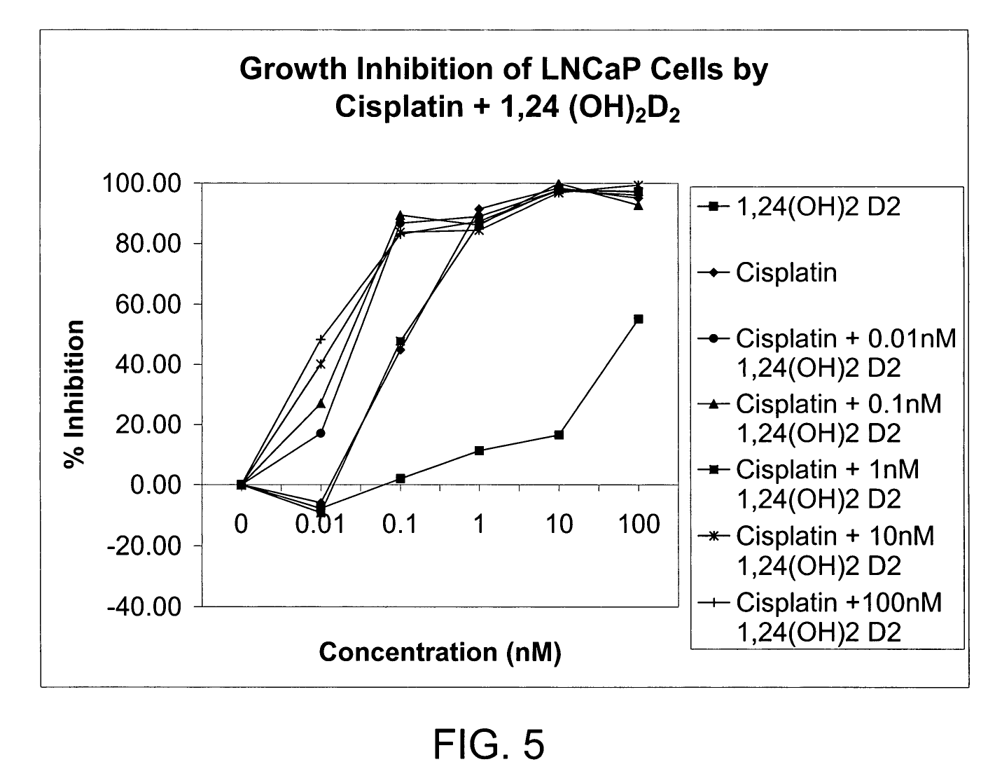

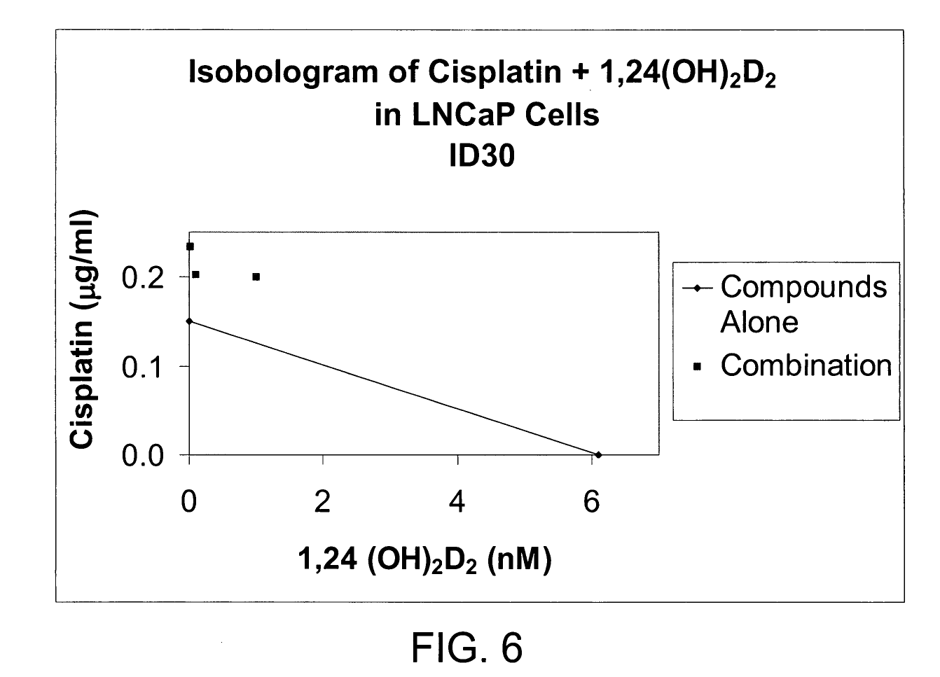

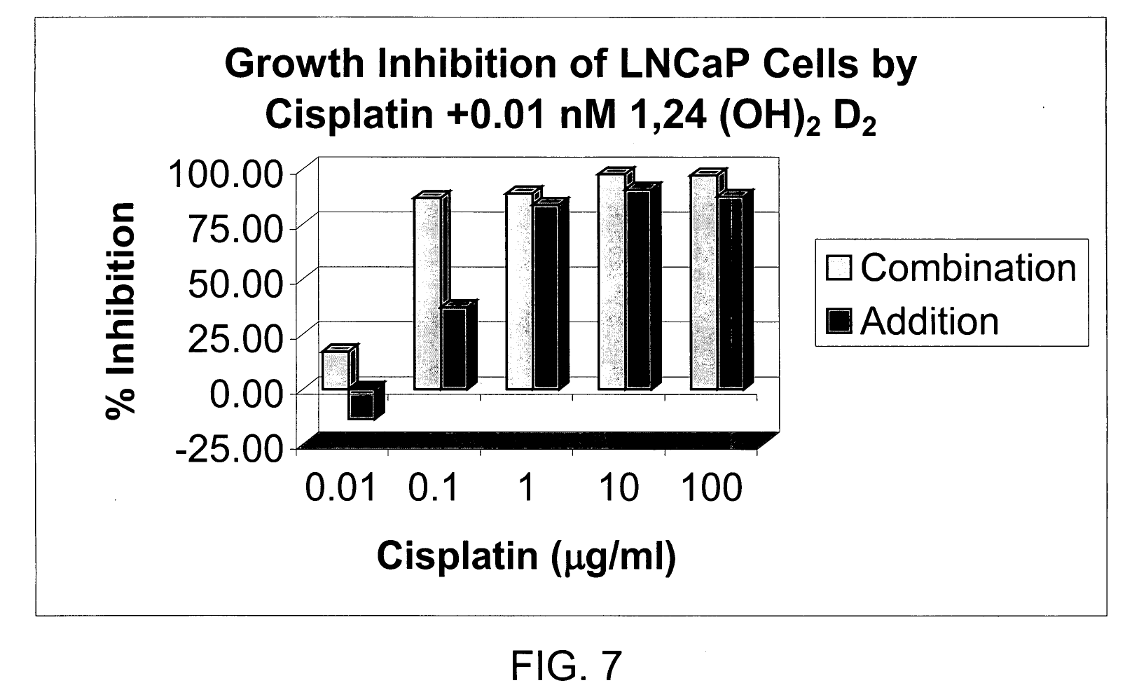

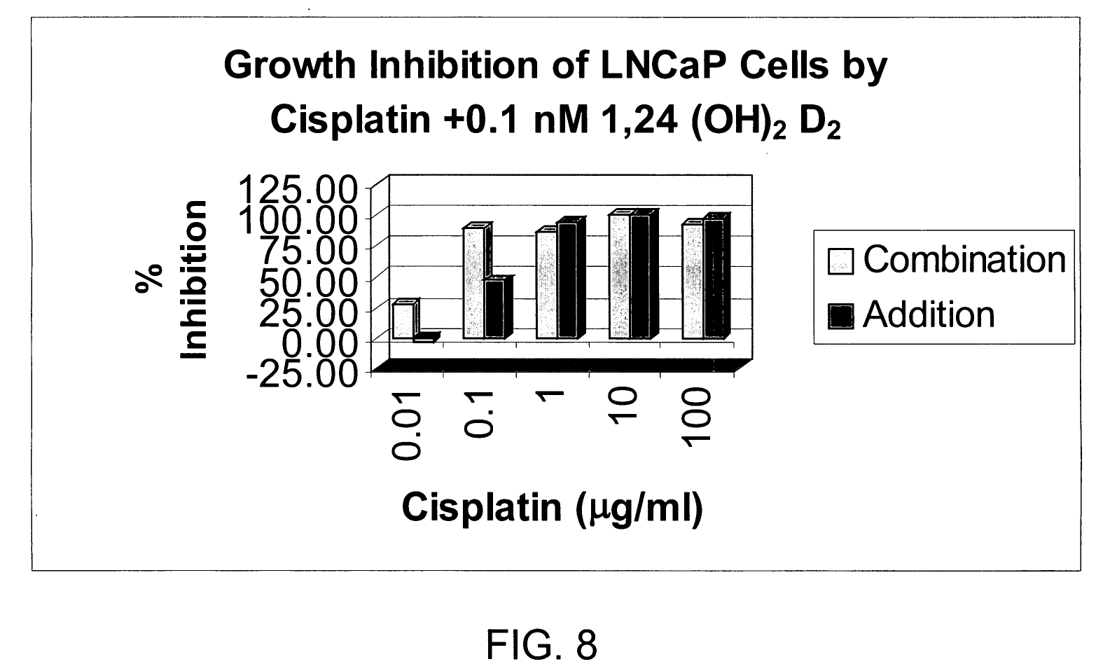

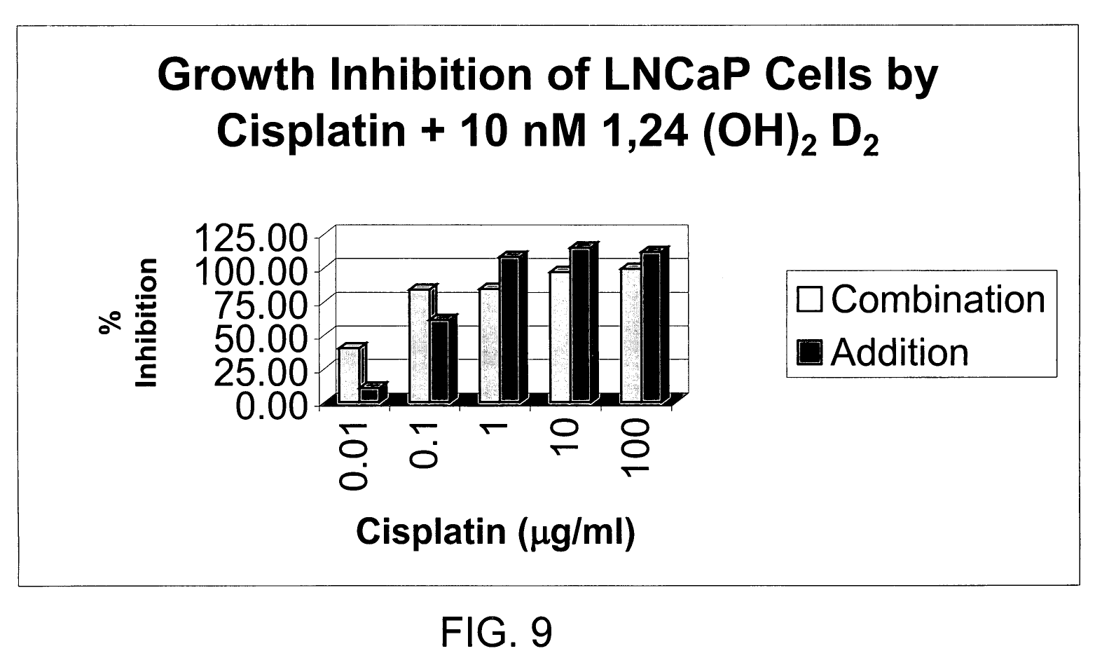

LNCap cells were plated in 96-well plates in triplicate and allowed to grow 48 hours. The medium was removed and replaced with medium containing vehicle (Ethanol), 1,24(OH)₂D₂ in various concentrations, and cisplatin in various concentrations. Cells were allowed to grow for an additional 6 days with media changed on day 3. Cell number was then determined by a colorimetric MTS assay and expressed as a % of change from control cells grown in vehicle only. FIG. 5 shows the percent inhibition of LNCaP cells of cisplatin alone or in combination with various concentrations of 1,24(OH)₂D₂. ID30 values (dose required to inhibit proliferation by 30%) were calculated to compare growth inhibitory effects of the compounds alone and in combination. Isobologram analysis was used to characterize the interaction between 1,24(OH)₂D₂ and cisplatin as synergistic, additive, or antagonistic. The isobologram is shown in FIG. 6. In FIGS. 7-9 the addition columns show the amount of inhibition predicted if the combination of cisplatin and 1,24(OH)₂D₂ simply had an additive effect on each other. The growth inhibition chart of FIG. 7 shows that the combination of cisplatin in concentrations of 0.01 μg/mL, 0.1 μg/mL, 1 μg/mL, 10 μg/mL and 100 μg/mL with 0.01 nM of 1,24(OH)₂D₂ produces additive to mild synergistic growth inhibition. The growth inhibition chart of FIG. 8 shows that the combination of cisplatin in concentrations of 0.01 μg/mL, and 0.1 μg/mL with 0.1 nM of 1,24(OH)₂D₂ produces additive to mild synergistic growth inhibition. The growth inhibition chart of FIG. 9 shows that the combination of cisplatin in concentrations of 0.01 μg/ml and 0.1 with 0.10 nM of 1,24(OH)₂D₂ produces additive to mild synergistic growth inhibition.

### EXAMPLE 11

### Growth Inhibition of LNCaP Cells by Busulfan and 1,24(OH)₂D₂

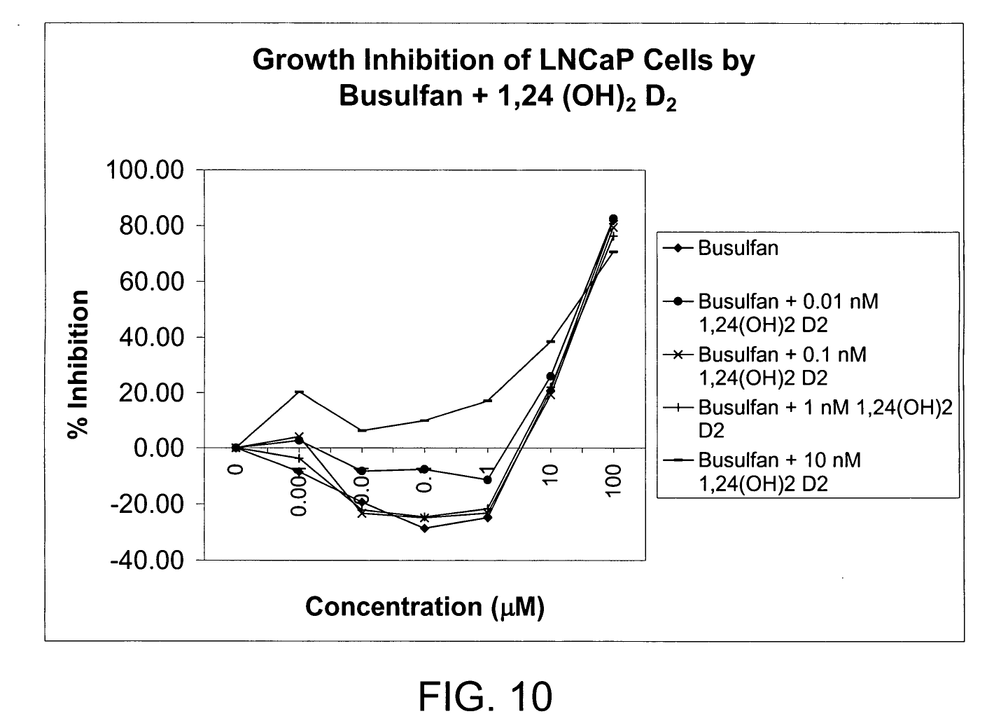

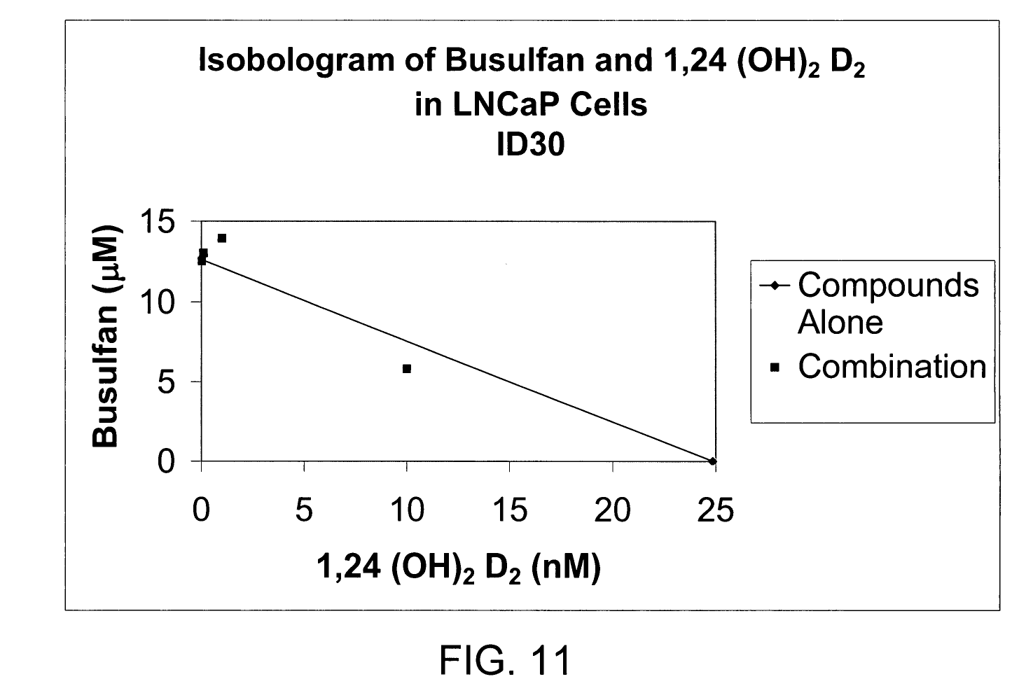

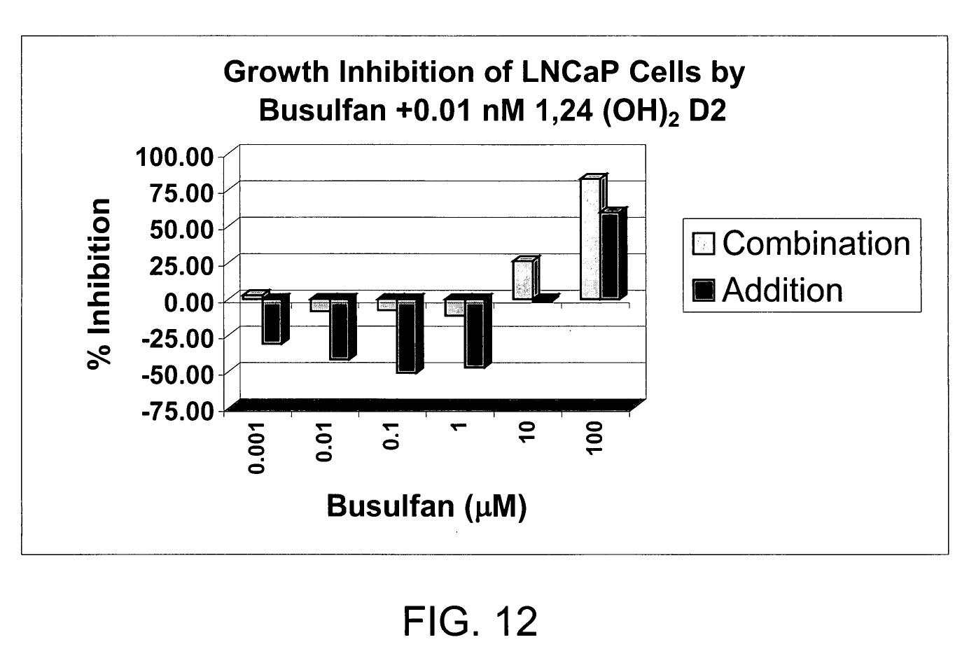

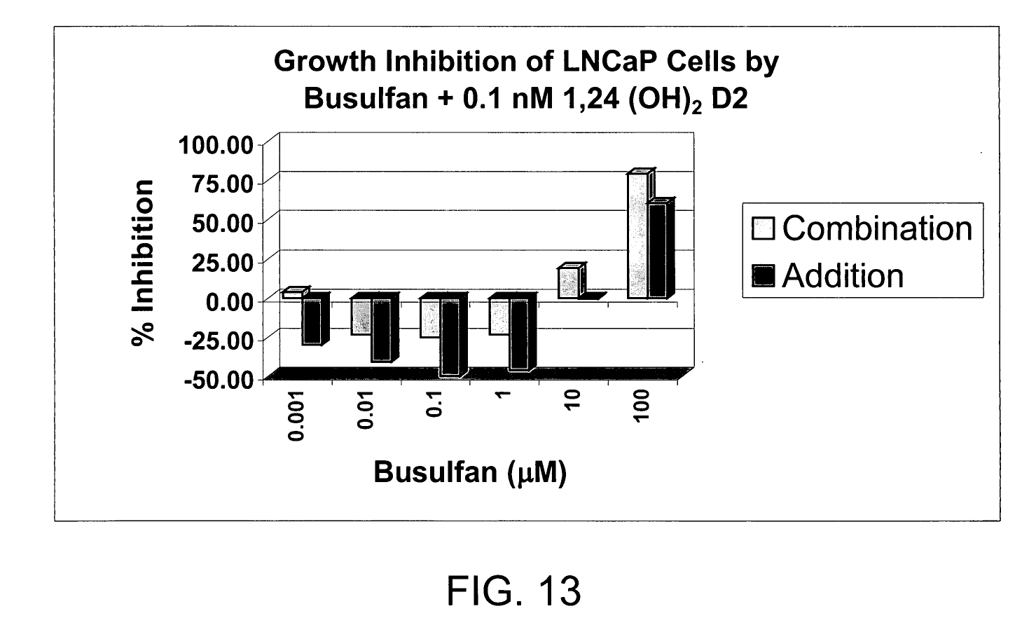

LNCap cells were plated in 96-well plates in triplicate and allowed to grow 48 hours. The medium was removed and replaced with medium containing vehicle (Ethanol), 1,24(OH)₂D₂ in various concentrations, and busulfan in various concentrations. Cells were allowed to grow for an additional 6 days with media changed on day 3. Cell number was then determined by a colorimetric MTS assay and expressed as a % of change from control cells grown in vehicle only. FIG. 10 shows the percent inhibition of LNCaP cells of busulfan alone or in combination with various concentrations of 1,24(OH)₂D₂. ID30 values (dose required to inhibit proliferation by 30%) were calculated to compare growth inhibitory effects of the compounds alone and in combination. Isobologram analysis was used to characterize the interaction between 1,24(OH)₂D₂ and busulfan as synergistic, additive, or antagonistic. The isobologram is shown in FIG. 11, and shows that busulfan in the concentration range of about 5 to 0.15 μM when combined with 1,24(OH)₂D₂ of various concentrations can provide a synergistic effect. This effect can also be seen in FIGS. 12-13. In FIGS. 12-13 the addition columns show the amount of inhibition predicted if the combination of busulfan and 1,24(OH)₂D₂ simply had an additive effect on each other. The growth inhibition chart of FIG. 12 shows that the combination of busulfan in concentrations of 10 μM and 100 μM with 0.01 nM of 1,24(OH)₂D₂ produces synergistic growth inhibition. The growth inhibition chart of FIG. 13 shows that the combination of busulfan in concentrations of 10 μM and 100 μM with 0.1 nM of 1,24(OH)₂D₂ produces synergistic growth inhibition.

### EXAMPLE 12

### Growth Inhibition of LNCaP Cells by Paclitaxel and 1,24(OH)₂D₂

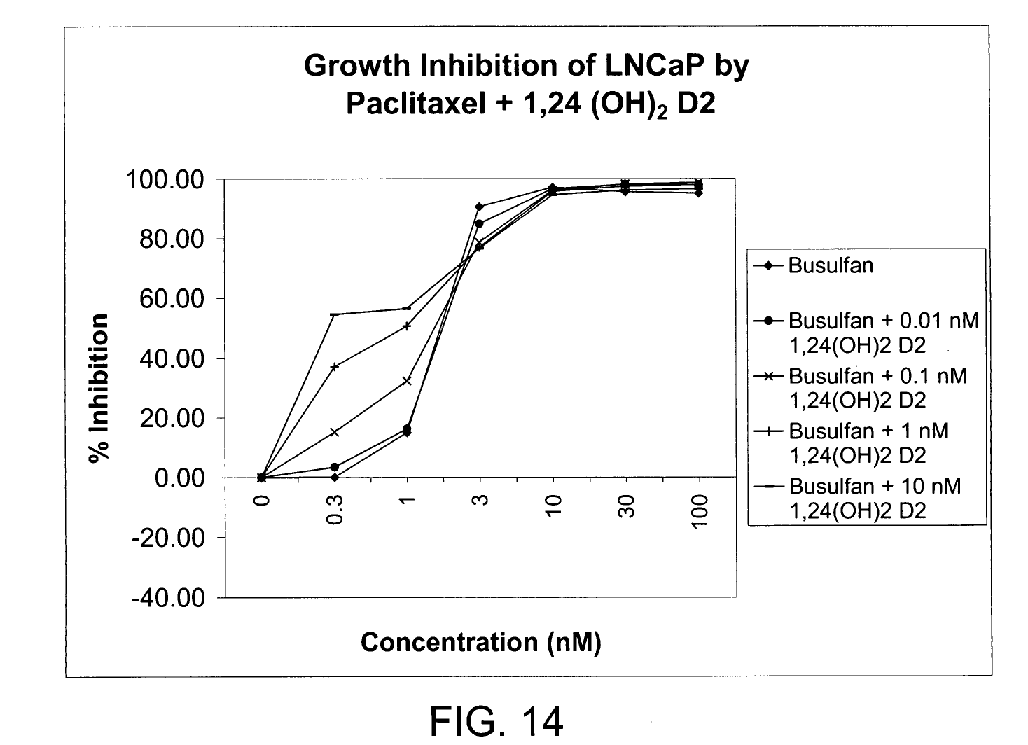

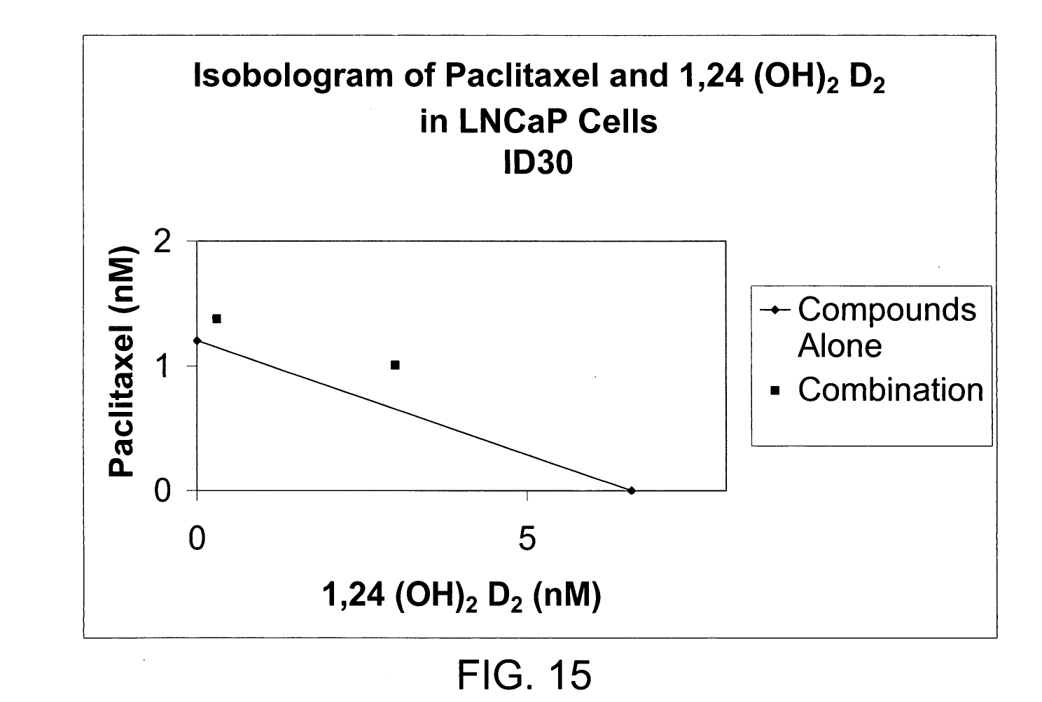

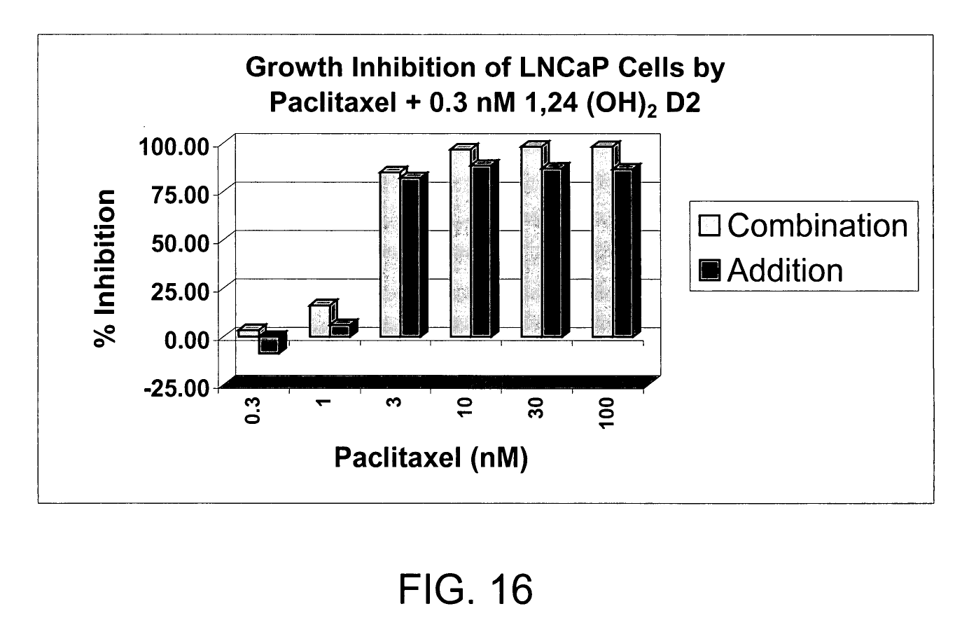

LNCap cells were plated in 96-well plates in triplicate and allowed to grow 48 hours. The medium was removed and replaced with medium containing vehicle (Ethanol), 1,24(OH)₂D₂ in various concentrations, and paclitaxel in various concentrations. Cells were allowed to grow for an additional 6 days with media changed on day 3. Cell number was then determined by a colorimetric MTS assay and expressed as a % of change from control cells grown in vehicle only. FIG. 14 shows the percent inhibition of LNCaP cells of paclitaxel alone or in combination with various concentrations of 1,24(OH)₂D₂. ID30 values (dose required to inhibit proliferation by 30%) were calculated to compare growth inhibitory effects of the compounds alone and in combination. Isobologram analysis was used to characterize the interaction between 1,24(OH)₂D₂ and paclitaxel as synergistic, additive, or antagonistic. The isobologram is shown in FIG. 15. In FIG. 16 the addition columns show the amount of inhibition predicted if the combination of paclitaxel and 1,24(OH)₂D₂ simply had an additive effect on each other. The growth inhibition chart of FIG. 16 shows that the combination of paclitaxel in concentrations of 0.3 nM, 1 nM, 3 nM, 10 nM, 20 nM and 100 nM with 0.3 nM of 1,24(OH)₂D₂ produces additive to mild synergistic growth inhibition.

### EXAMPLE 13

### Growth Inhibition of LNCaP Cells by Etoposide and with 1,24(OH)₂D₂

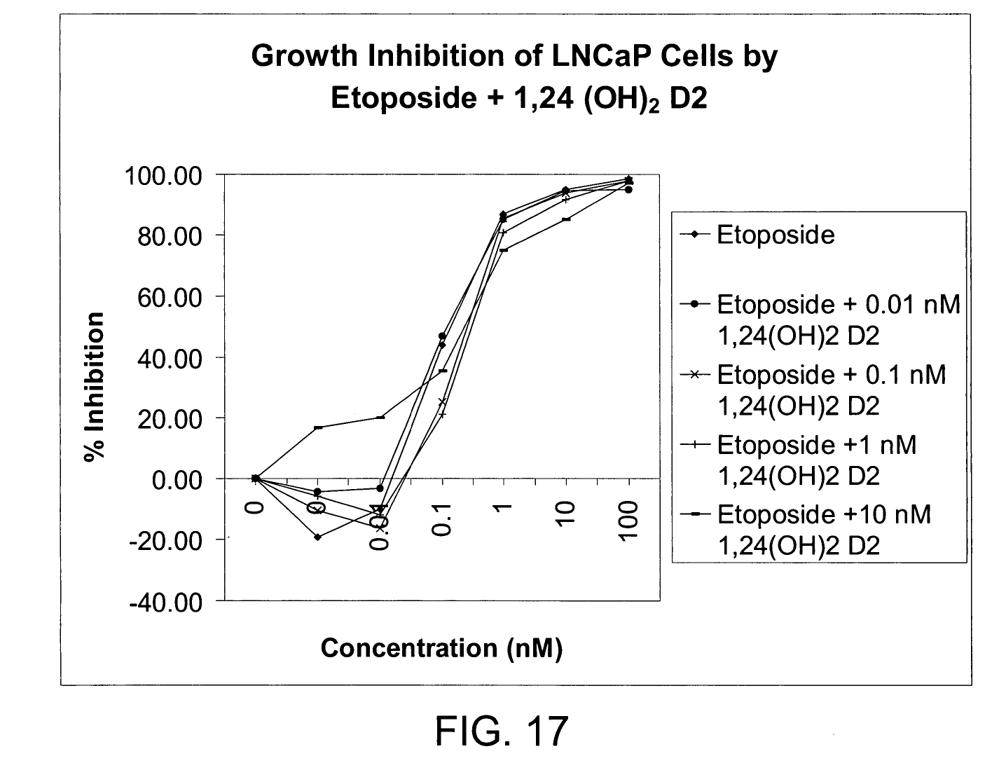

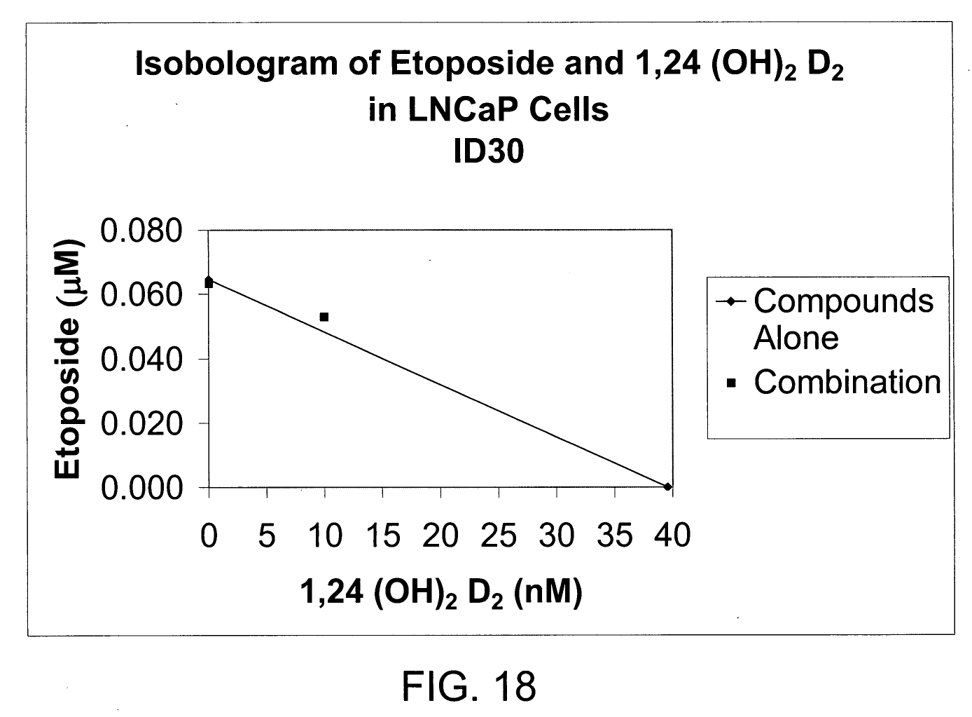

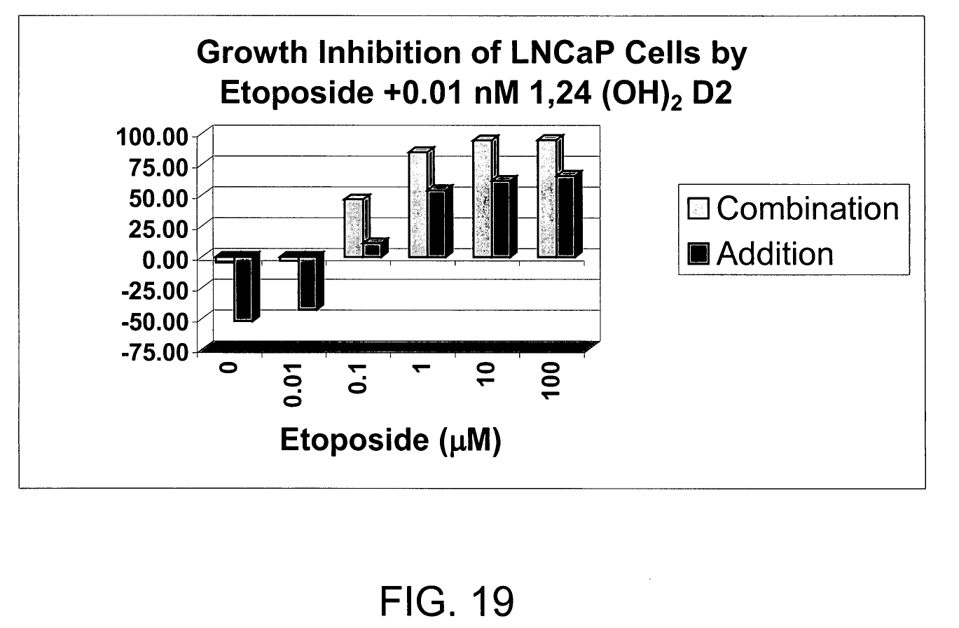

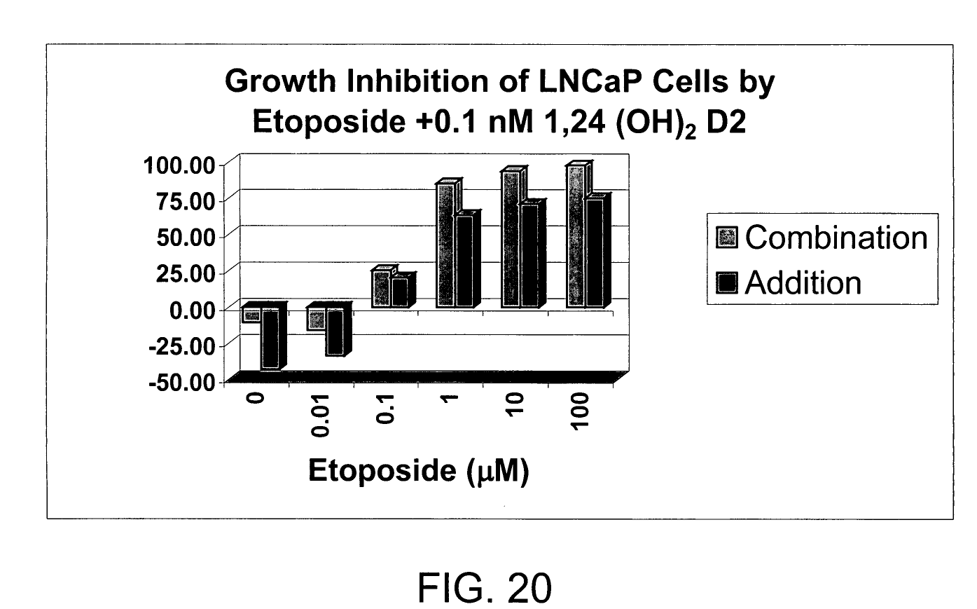

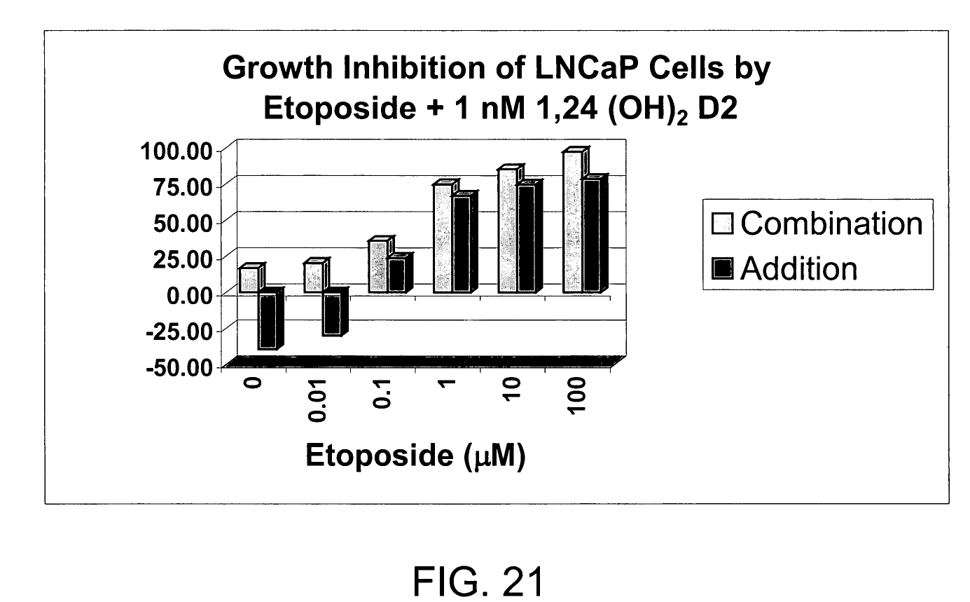

LNCap cells were plated in 96-well plates in triplicate and allowed to grow 48 hours. The medium was removed and replaced with medium containing vehicle (Ethanol), 1,24(OH)₂D₂ in various concentrations, and etoposide in various concentrations. Cells were allowed to grow for an additional 6 days with media changed on day 3. Cell number was then determined by a calorimetric MTS assay and expressed as a % of change from control cells grown in vehicle only. FIG. 17 shows the percent inhibition of LNCaP cells of etoposide alone or in combination with various concentrations of 1,24(OH)₂D₂. ID30 values (dose required to inhibit proliferation by 30%) were calculated to compare growth inhibitory effects of the compounds alone and in combination. Isobologram analysis was used to characterize the interaction between 1,24(OH)₂D₂ and etoposide as synergistic, additive, or antagonistic. The isobologram is shown in FIG. 18. In FIGS. 19-21 the addition columns show the amount of inhibition predicted if the combination of etoposide and 1,24(OH)₂D₂ simply had an additive effect on each other. The growth inhibition chart of FIG. 19 shows that the combination of etoposide in concentrations of 0.1 μM, 1 μM, 10 μM and 100 μM with 0.1 nM of 1,24(OH)₂D₂ produces synergistic growth inhibition. The growth inhibition chart of FIG. 20 shows that the combination of etoposide in concentrations of 0.1 μM, 1 μM, 10 μM and 100 μM with 0.1 μM of 1,24(OH)₂D₂ produces synergistic growth inhibition. The growth inhibition chart of FIG. 20 shows that the combination of etoposide in concentrations of 0.01 μM, 0.1 μM, 1 μM, 10 μM and 100 μM with 1 nM of 1,24(OH)₂D₂ produces synergistic growth inhibition.

### EXAMPLE 14

### Growth Inhibition of LNCaP Cells by 5-Fluorouracil and with 1,24(OH)₂D₂

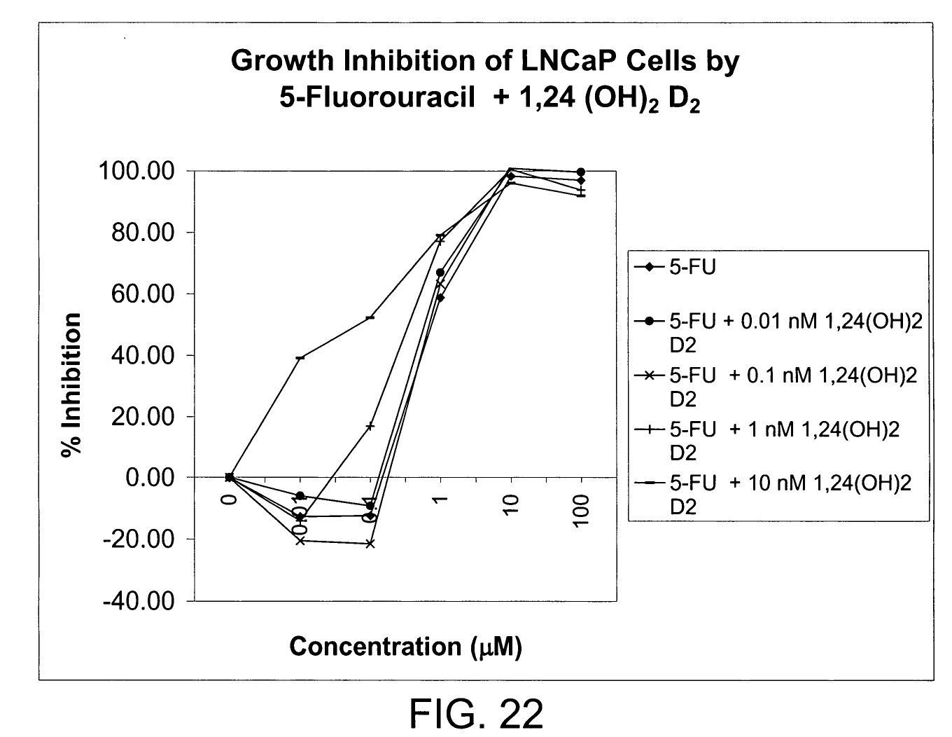

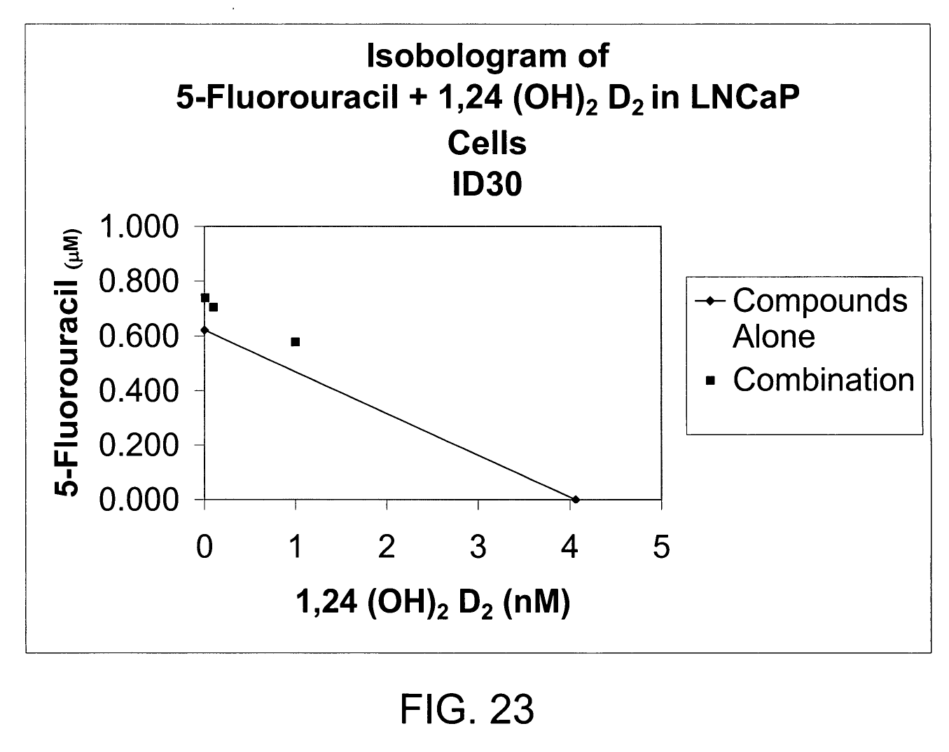

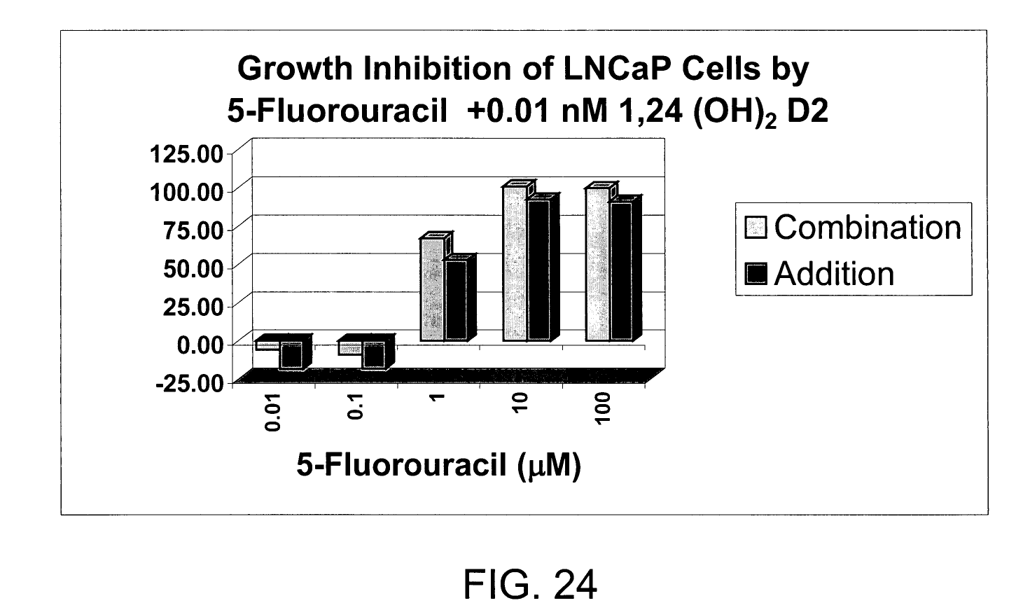

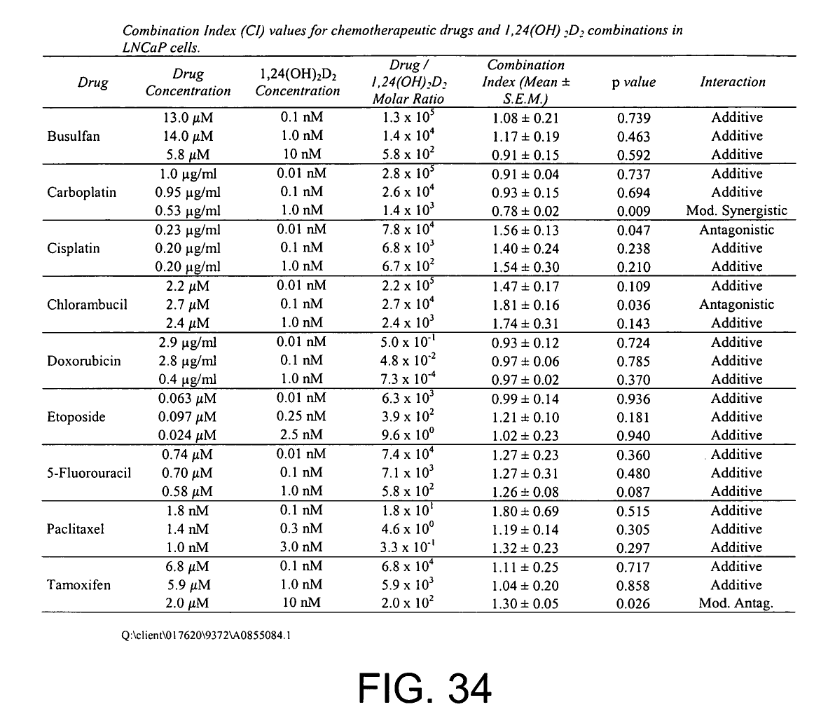

LNCap cells were plated in 96-well plates in triplicate and allowed to grow 48 hours. The medium was removed and replaced with medium containing vehicle (Ethanol), 1,24(OH)₂D₂ in various concentrations, and 5-Fluorouracil in various concentrations. Cells were allowed to grow for an additional 6 days with media changed on day 3. Cell number was then determined by a calorimetric MTS assay and expressed as a % of change from control cells grown in vehicle only. FIG. 22 shows the percent inhibition of LNCaP cells of 5-Fluorouracil alone or in combination with various concentrations of 1,24(OH)₂D₂. ID30 values (dose required to inhibit proliferation by 30%) were calculated to compare growth inhibitory effects of the compounds alone and in combination. Isobologram analysis was used to characterize the interaction between 1,24(OH)₂D₂ and 5-Fluorouracil as synergistic, additive, or antagonistic. The isobologram is shown in FIG. 23. In FIG. 34 the addition columns show the amount of inhibition predicted if the combination of 5-Fluorouracil and 1,24(OH)₂D₂ simply had an additive effect on each other. The growth inhibition chart of FIG. 24 shows that the combination of 5-Fluorouracil in concentrations of 1 μM, 10 μM and 100 μM with 0.01 nM of 1,24(OH)₂D₂ produces additive to mild synergistic growth inhibition.

### EXAMPLE 15

### Growth Inhibition of LNCaP Cells by Tamoxifen and With 1,24(OH)₂D₂

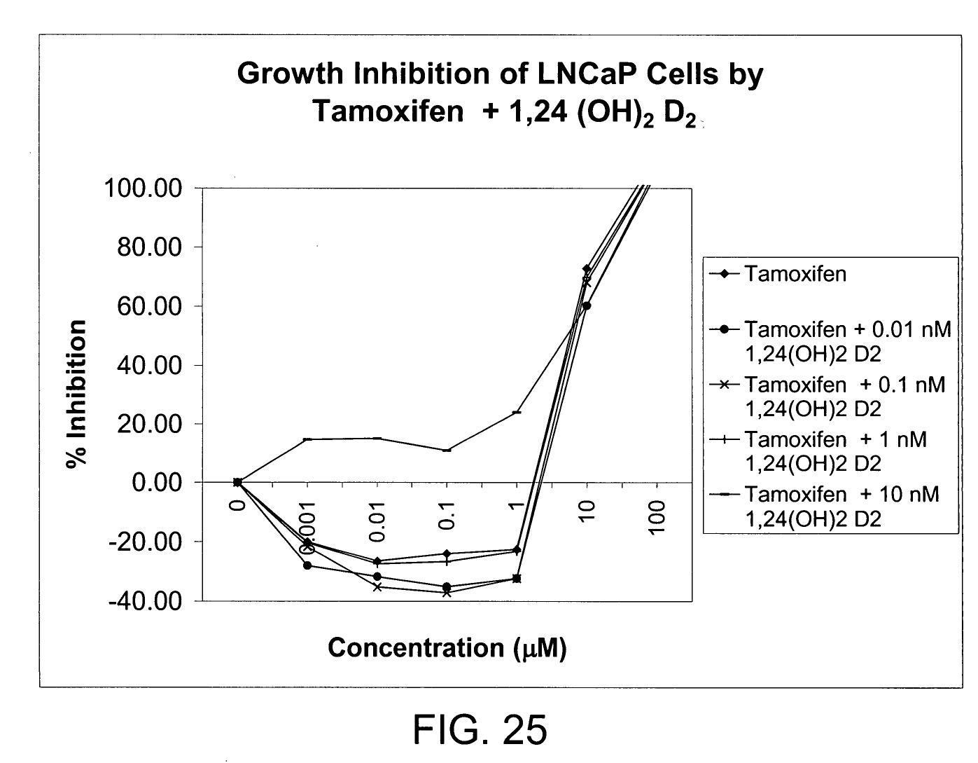

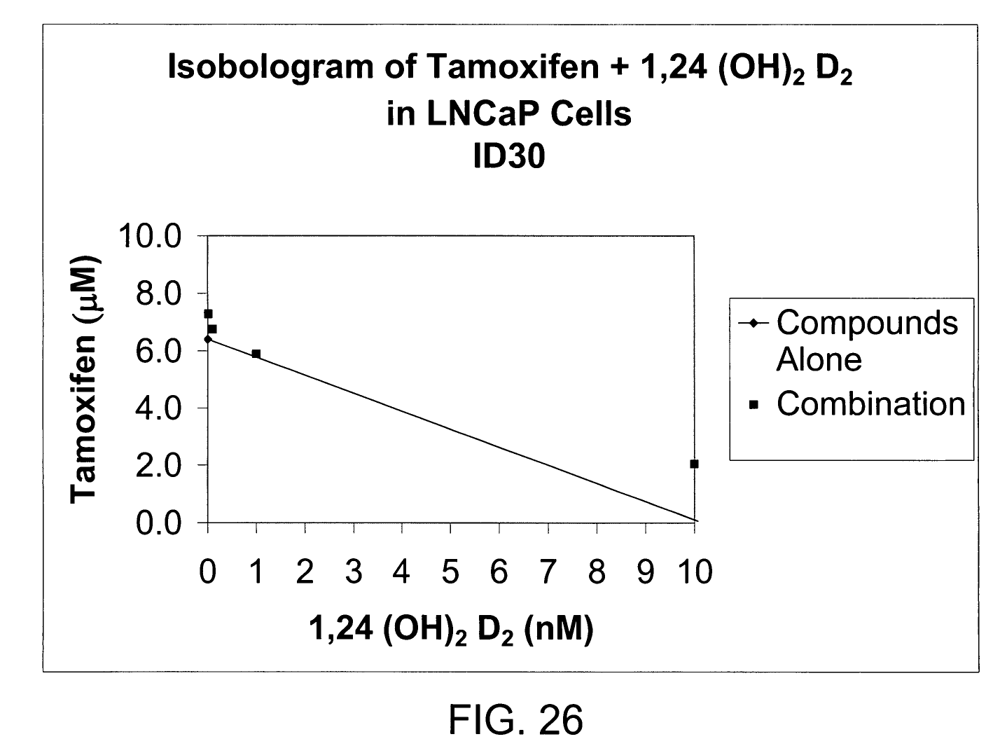

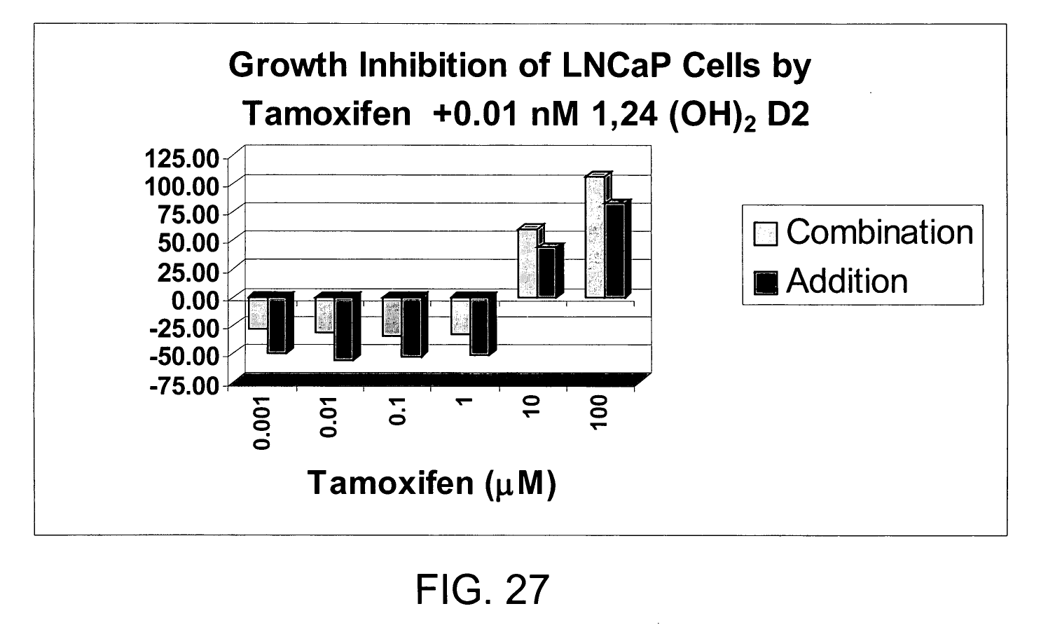

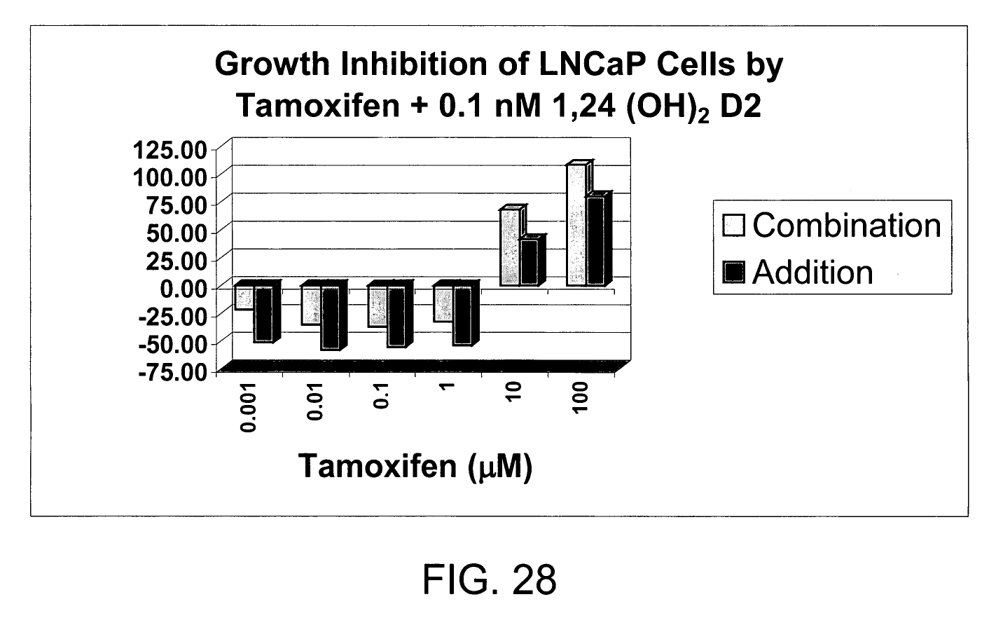

LNCap cells were plated in 96-well plates in triplicate and allowed to grow 48 hours. The medium was removed and replaced with medium containing vehicle (Ethanol), 1,24(OH)₂D₂ in various concentrations, and tamoxifen in various concentrations. Cells were allowed to grow for an additional 6 days with media changed on day 3. Cell number was then determined by a colorimetric MTS assay and expressed as a % of change from control cells grown in vehicle only. FIG. 25 shows the percent inhibition of LNCaP cells of tamoxifen alone or in combination with various concentrations of 1,24(OH)₂D₂. ID30 values (dose required to inhibit proliferation by 30%) were calculated to compare growth inhibitory effects of the compounds alone and in combination. Isobologram analysis was used to characterize the interaction between 1,24(OH)₂D₂ and tamoxifen as synergistic, additive, or antagonistic. The isobologram is shown in FIG. 26, and shows that tamoxifen in the concentration range of about 0 to 10 μM when combined with 1,24(OH)₂D₂ of various concentrations can provide a synergistic effect. This effect can also be seen in FIGS. 27-28. In FIGS. 27-28 the addition columns show the amount of inhibition predicted if the combination of tamoxifen and 1,24(OH)₂D₂ simply had an additive effect on each other. The growth inhibition chart of FIG. 27 shows that the combination of tamoxifen in concentrations of 10 μM and 100 μM with 0.01 nM of 1,24(OH)₂D₂ produces synergistic growth inhibition. The growth inhibition chart of FIG. 28 shows that the combination of tamoxifen in concentrations of 10 μM and 100 μM with 0.1 nM of 1,24(OH)₂D₂ produces synergistic growth inhibition.

### EXAMPLE 16

### Growth Inhibition of LNCaP Cells by Doxorubicin and with 1,25(OH)₂D₂

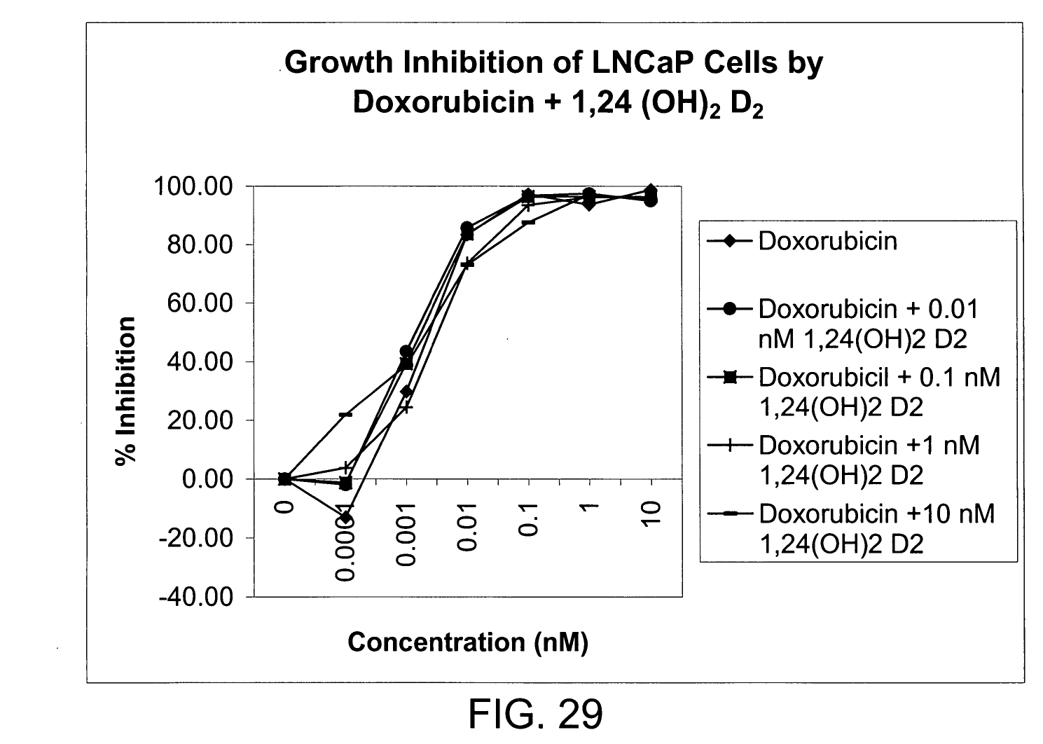

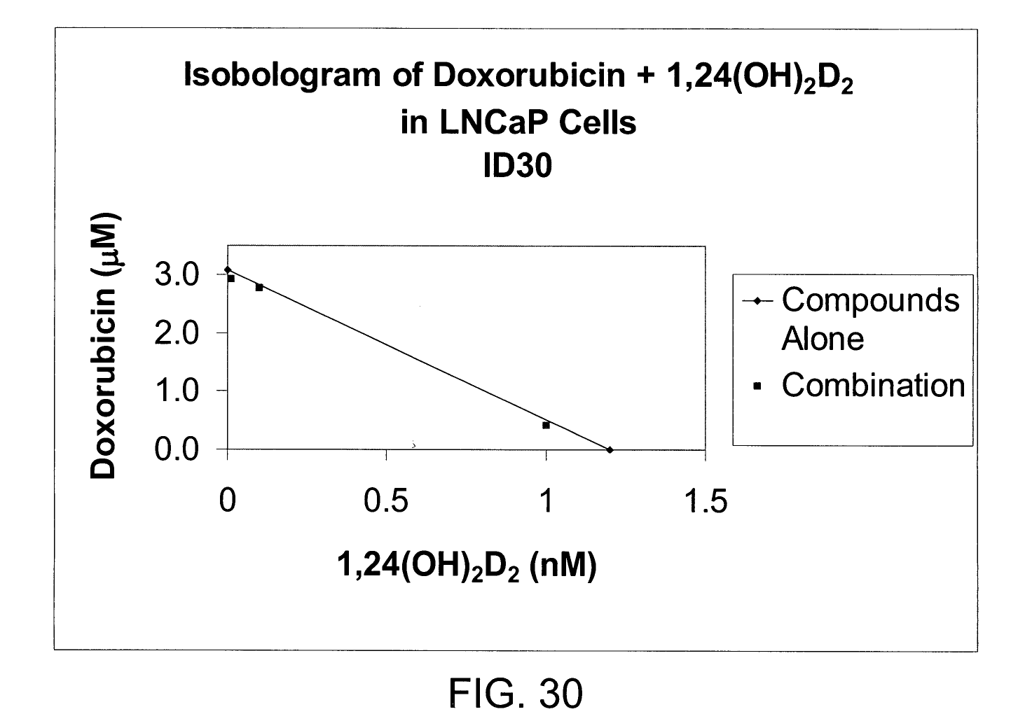

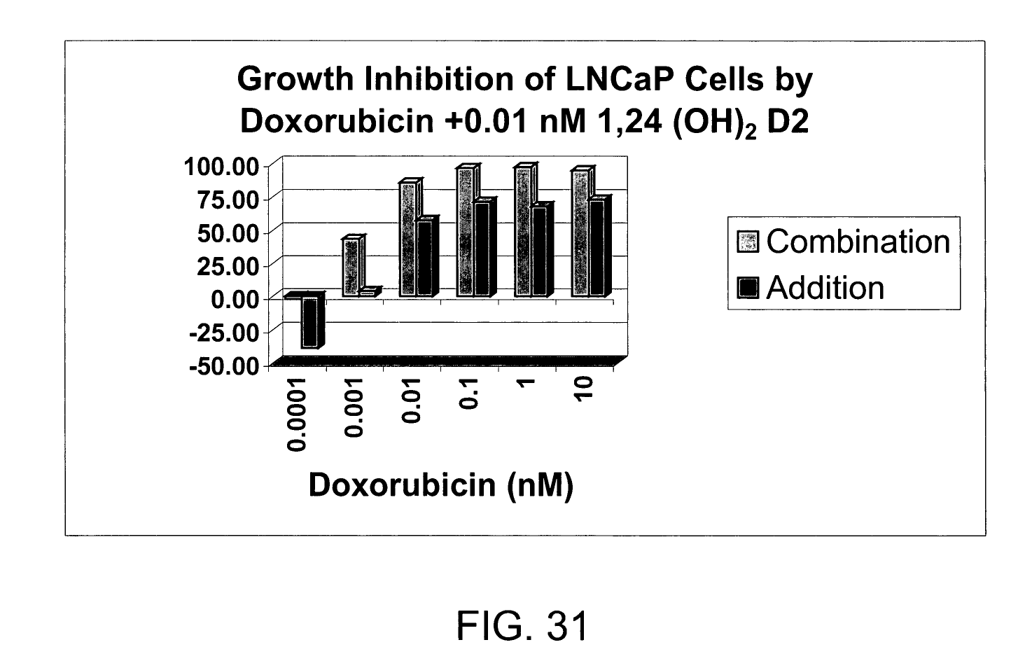

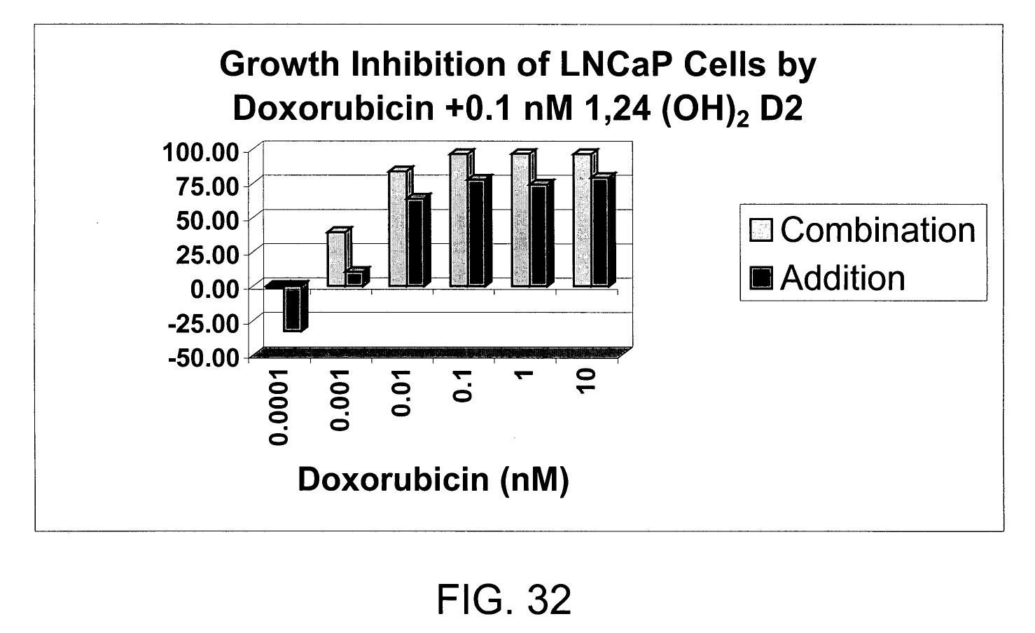

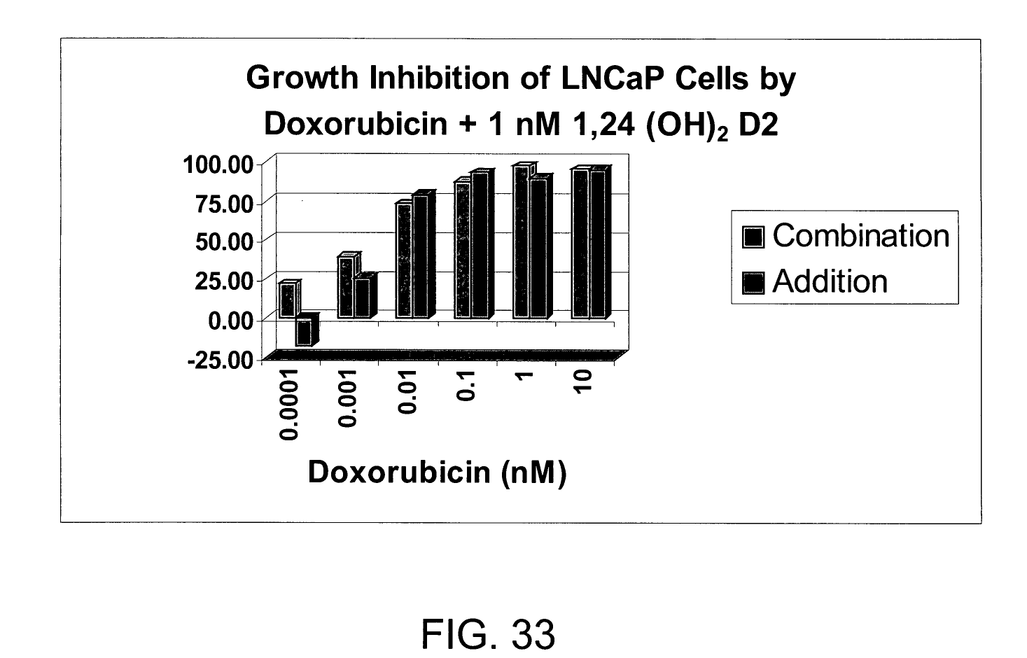

LNCap cells were plated in 96-well plates in triplicate and allowed to grow 48 hours. The medium was removed and replaced with medium containing vehicle (Ethanol), 1,24(OH)₂D₂ in various concentrations, and doxorubicin in various concentrations. Cells were allowed to grow for an additional 6 days with media changed on day 3. Cell number was then determined by a colorimetric MTS assay and expressed as a % of change from control cells grown in vehicle only. FIG. 29 shows the percent inhibition of LNCaP cells of doxorubicin alone or in combination with various concentrations of 1,24(OH)₂D₂. ID30 values (dose required to inhibit proliferation by 30%) were calculated to compare growth inhibitory effects of the compounds alone and in combination. Isobologram analysis was used to characterize the interaction between 1,24(OH)₂D₂ and doxorubicin as synergistic, additive, or antagonistic. The isobologram is shown in FIG. 30. FIGS. 31-33 show that in certain concentrations, doxorubicin can have a synergistic effect when combined with 1,24(OH)₂D₂. In FIGS. 31-33, the addition columns show the amount of inhibition predicted if the combination of doxorubicin and 1,24(OH)₂D₂ simply had an additive effect on each other. The growth inhibition chart of FIG. 31 shows that the combination of doxorubicin in concentrations of 0.001 nM, 0.01 nM, 0.1 nM, 1 nM and 10 nM with 0.01 nM of 1,24(OH)₂D₂ produces synergistic growth inhibition. The growth inhibition chart of FIG. 32 shows that the combination of doxorubicin in concentrations of 0.001 nM, 0.01 nM, 0.1 nM, 1 nM and 10 nM with 0.1 nM of 1,24(OH)₂D₂ produces synergistic growth inhibition. The growth inhibition chart of FIG. 33 shows that the combination of doxorubicin in concentrations of 0.0001 nM, and 0.001 nM with 1 nM of 1,24(OH)₂D₂ produces additive to mild synergistic growth inhibition.

### EXAMPLE 17

### Combination Index (CI) Values for Chemotherapeutic Drugs and 1,24(OH)₂D₂ Combinations in LNCaP Cells

As shown in FIG. 34, 1,24(OH)₂D₂ was dosed in combination with individual anticancer agents at several different molar ratios as described in Example 7. The degree of interaction between two drugs was calculated using the combination index, according to the isobologram equation:
CI=d₁/D₁+d₂/D_{2.}

In this equation, d₁, and d₂ represent the doses of drug 1 and drug 2 that, when given in combination, produce a specific response, and D₁ and D₂ represent the doses of drug 1 and drug 2 when given individually, produce the same effect. Drug interactions were classified according to the following criteria:

| Combination Index (CI) | Drug Interaction Description |
|---|---|
| <0.1 | Very Strong Synergism |
| 0.1-0.3 | Strong Synergism |
| 0.3-0.7 | Synergism |
| 0.7-0.85 | Moderate Synergism |
| 0.85-0.90 | Slight Synergism |
| 0.90-1.10 | Additive |
| 1.10-1.20 | Slight Antagonism |
| 1.20-1.45 | Moderate Antagonism |
| 1.45-3.3 | Antagonism |
| 3.3-10 | Strong Antagonism |
| >10 | Very Strong Antagonism |

Multiple trials were run to determine a p value for the combination index for the drug combinations Degree of interaction is defined as significant at p<0.075.

While the present invention has now been described and exemplified with some specificity, those skilled in the art will appreciate the various modifications, including variations, additions, and omissions, that may be made in what has been described. Accordingly, it is intended that these modifications also be encompassed by the present invention and that the scope of the present invention be limited solely by the broadest interpretation lawfully accorded the appended claims.

All patents, publications and references cited herein are hereby fully incorporated by reference. In case of conflict between the present disclosure and incorporated patents, publications and references, the present disclosure should control.

1. A method of synergistically inhibiting the growth of human prostatic neoplastic or hyperplastic cells, comprising contacting the cells with a first composition which comprises 1α,24-dihydroxyvitamin d₂ and a second composition which comprises carboplatin.

2. The method of claim 1, wherein the first and second compositions are administered to a human cancer patient.

3. The method of claim 2 wherein the first and second compositions are co-administered.

4. The method of claim 2 wherein the first and second compositions are administered in a daily regimen.

5. The method of claim 2 wherein the first and second compositions are administered in a episodic regimen.

6. The method of claim 2 wherein the first composition is administered intravenously.

7. The method of claim 2 wherein the first composition is administered orally.

8. The method of claim 2 wherein the first composition is administered amount in an amount of 0.01 μg to 400 μg.

9. A pharmaceutical combination for the inhibition of the hyperproliferative activity of human prostatic neoplastic or hyperplastic cells which comprises a therapeutically effective dose of a synergistic combination of a first agent which is 1α,24-dihydroxyvitamin D₂ and a second agent which is carboplatin.

10. A pharmaceutical combination comprising a first agent which is 1α,24-dihydroxyvitamin D₂ and a second agent which is carboplatin, wherein the first and second agents have synergistic properties for inhibiting the hyperproliferative activity of human prostatic neoplastic or hyperplastic cells.

11. A method of additively inhibiting the growth of human prostatic neoplastic or hyperplastic cells, comprising contacting the cells with a first composition comprising 1α,24-dihydroxyvitamin D₂ and a second composition which comprises an agent selected from the group consisting of carboplatin, doxorubicin, chlorambucil, busulfan, cisplatin, paclitaxel, etoposide, 5-flurouracil, and tamofixen.

12. The method of claim 111 wherein the agent is carboplatin.

13. The method of claim 111 wherein the agent is doxorubicin.

14. The method of claim 11 wherein the agent is chlorambucil.

15. The method of claim 11 wherein the agent is busulfan.

16. The method of claim 11 wherein the agent is cisplatin.

17. The method of claim 11 wherein the agent is paclitaxel.

18. The method of claim 11 wherein the agent is etoposide.

19. The method of claim 11 wherein the agent is 5-flurouracil.

20. The method of claim 11 wherein the agent is tamofixen.

21. The method of claim 11, wherein the first and second compositions are administered to a human cancer patient.

22. The method of claim 21 wherein the first and compositions agents are co-administered.

23. The method of claim 21 wherein the first and second compositions are administered in a daily regimen.

24. The method of claim 21 wherein the first and second compositions are administered in a episodic regimen.

25. The method of claim 21 wherein the first composition is administered intravenously.

26. The method of claim 21 wherein the first composition is administered orally.

27. The method of claim 21 wherein the first composition is administered amount in an amount of 0.01 μg to 400 μg.

28. A pharmaceutical combination for the inhibition of growth of human prostatic neoplastic or hyperplastic cells which comprises a therapeutically effective dose of an additive combination of a first agent which is 1α,24-dihydroxyvitamin D₂ and a second agent selected from the group consisting of carboplatin, doxorubicin, chlorambucil, busulfan, cisplatin, paclitaxel, etoposide, 5-flurouracil, and tamofixen.

29. A pharmaceutical combination comprising a first agent which is 1α,24-dihydroxyvitamin D₂ and a second agent which is selected from the group consisting of carboplatin, doxorubicin, chlorambucil, busulfan, cisplatin, paclitaxel, etoposide, 5-flurouracil, and tamofixen, wherein the first and second agents have additive properties for inhibiting the hyperproliferative activity of human prostatic neoplastic or hyperplastic cells.
